(19) United States
(12) Patent Application Publication
(10) Pub. No.: US 2026/0224523 A1
(43) Pub. Date: Aug. 6, 2026

(54) Induction of Proteasomal Activity in Mammalian Cells by Lifespan-extending tRNA Synthetase Inhibitors

(71) Applicant: UNM RAINFOREST INNOVATIONS, Albuquerque, NM (US)

(72) Inventors: MARK A. McCORMICK, ALBUQUERQUE, NM (US); BLAISE L. MARINER, TUCSON, AZ (US)

(21) Appl. No.: 19/179,017

(22) Filed: Apr. 15, 2025

Related U.S. Application Data
(60) Provisional application No. 63/637,460, filed on Apr. 23, 2024.

(51) Int. Cl. A61K 31/365; A61K 31/351; A61K 31/352; A61K 31/517; A61K 31/549; A61K 31/55; A61K 31/69; A61K 38/17; A61K 38/45; A61K 39/00; A61K 39/395; A61K 45/06

(52) U.S. Cl. CPC A61K 31/365; A61K 31/351; A61K 31/352; A61K 31/517; A61K 31/549; A61K 31/55; A61K 31/69; A61K 38/1709; A61K 38/45; A61K 39/0005; A61K 39/3955; A61K 45/06; C12Y 207/11001

(57) ABSTRACT

The present invention is directed to methods for treating, delaying, inhibiting, ameliorating or reducing the likelihood of neurodegenerative disease in a patient in need or at risk using tRNA synthetase inhibitors including borrelidin, mupirocin, tavaborole, halofuginone, LysRS-IN-2, REP 3132 (CRS-3132), REP8839 and mixtures thereof, among others as described herein.

### RELATED APPLICATIONS AND FEDERAL GRANT SUPPORT

This application claims the benefit of priority of U.S. provisional application Ser. No. 63/637,460, filed Apr. 23, 2024, the entire contents of which application are incorporated by reference herein.

This invention was made with government support under P20 GM121176 awarded by the National Institutes of Health. The government has certain rights in the invention.

### FIELD OF THE INVENTION

The present invention is directed to methods for treating, delaying, inhibiting, ameliorating or reducing the likelihood of neurodegenerative disease including amyotrophic lateral sclerosis, multiple sclerosis, Parkinson's disease, Alzheimer's disease, Huntington's disease, multiple system atrophy, tauopathies (Pick's disease, supranuclear palsy and corticobasal degeneration) and prion diseases (Creutzfeldt-Jacob disease (CJD), variant Creutzfeldt-Jacob disease (VCJD), variably protease-sensitive prionopathy (VPSPr), Gerstmann-Sträussler-Scheinker disease (GSS), Kuru and Fatal insomnia (FI)) in a patient in need or at risk using tRNA synthetase inhibitors including borrelidin, mupirocin, tavaborole, halofuginone, LysRS-IN-2, REP 3132(CRS-3132), REP 8839 and mixtures thereof, among others as described herein.

### BACKGROUND AND OVERVIEW OF THE INVENTION

The ubiquitin proteasome system (UPS) is essential for the turnover of many polypeptides in eukaryotes, but is especially responsible for the turnover of the dysfunctional, mutated, misfolded, or damaged proteins [1]. The UPS has been hypothesized as a high-impact target to treat many diseases such as Huntington's disease, Alzheimer's disease, and Parkinson's disease, among others [2-7]. Increased UPS activity has also been hypothesized to improve an organism's ability to maintain protein homeostasis, a hallmark of aging [3,4,8-15]. Drugs that activate or enhance proteasome activity are rare, especially in comparison to proteasome inhibitors [6].

Activating transcription factor 4 (ATF4) is a conserved transcription factor of recent interest in its wide variety of diseases such as neurodegeneration, diabetes, cancer, and skeletal muscle aging [16-27]. The Atf4 orthologs GCN4/atf-4 have been linked to increased lifespan in both the budding yeast S. cerevisiae and the nematode C. elegans [18,21,28-31]. Further, long-lived mice have been shown to have elevated levels of ATF4 [32,33].

There are four kinases in vertebrates that are known to up-regulate ATF4 translation through the integrated stress response (ISR), which can be activated in response to endoplasmic reticulum stress, amino acid deprivation, the presence of double stranded RNA, and, in erythroid cells, heme deficiency [16]. These four kinases phosphorylate the alpha subunit of eukaryotic initiation factor 2 (eIF2α), an essential protein in the formation of the translation pre-initiation complex in eukaryotes. This eIF2α phosphorylation results in the the delay in translational re-initiation and subsequent ATF4 translation [16,34]. General control non-derepressible kinase 2 (GCN2) is an eIF 2α kinase and the activator of the amino acid response leg of the ISR [35]. GCN2 autophosphorylates and activates upon the kinase's binding to uncharged tRNA [20,34,36,37].

There are two important modes of protein degradation in the cell. First, autophagy is a degradation process in which cellular components, such as proteins and organelles, are delivered to the degradative organelles for breakdown and re-purposing of macromolecules, like amino acids [38]. There are many different flavors of autophagy, such as, but not limited to, mitophagy, selective autophagy, and ribophagy, all of which are only starting to be understood especially in the context of aging [39-43]. Macroautophagy, (hereafter ‘autophagy’), is the most widely-studied, and has been established as an important biological process in many long-lived organisms [28,40,44-48]. Autophagy genes and their role in aging and disease phenotypes are widely studied and have been recently reviewed [49]. ATF4 is known to aid in the induction of autophagy through its role as a transcription factor [50-57]. Second, the UPS degrades “tagged” or poly-ubiquitinated proteins [1]. In contrast, autophagy is a form of bulk component recycling, while the proteasome turns over proteins on a more individual level, resulting in the proteasome's tight connection to the regulation of many cellular responses [1]. Increased proteasomal capacity has been found to be responsible for some extremely long-lived phenotypes in yeast, worms and flies, indicating that further research exploring this process in mammals is of great interest to the aging field [3,4,8-10,18]. Altogether, the promotion of these two processes to increase protein degradation has been shown to increase health in many models, underscoring the importance of an organism's ability to maintain protein homeostasis in healthy aging [3,5,40,58-63].

Although there is great interest in the treatment potential of UPS activators, there are few known pharmacological agents that can do so [5]. In the present application, the inventors show that seven different tRNA synthetase inhibitors can dramatically induce proteasome activity in an Atf4-dependent manner in mammalian cells in vitro. We also show that these same drugs can upregulate proteasomal activity at the same doses, as well as macroautophagy, suggesting these drugs'potential to treat important diseases of aging characterized by protein aggregation in vivo. As we have recently shown that some of these same compounds dramatically increase healthy wild-type lifespan in multiple model organisms, this also leaves open the possibility that these potential treatments for known and significant diseases of aging might also act directly on aging itself [27].

### Brief Description of the Invention

In an embodiment, the invention is directed to a method for treating, inhibiting, delaying or reducing the likelihood of neurodegenerative disease in a patient in need or at risk for neurodegenerative disease comprising administering to said patient an effective amount of at least one tRNA Synthetase inhibitors.

In an embodiment, the neurodegenerative disease is amyotrophic lateral sclerosis, multiple sclerosis, Parkinson's disease, Alzheimer's disease, Huntington's disease, multiple system atrophy, tauopathies (Pick's disease, supranuclear palsy and corticobasal degeneration) and prion diseases (Creutzfeldt-Jacob disease (CJD), variant Creutzfeldt-Jacob disease (VCJD), variably protease-sensitive prionopathy (VPSPr), Gerstmann-Sträussler-Scheinker disease (GSS), Kuru or Fatal insomnia (FI)).

In embodiments, the neurodegenerative disease is amyotrophic lateral sclerosis, multiple sclerosis, Parkinson's disease, Alzheimer's disease or Huntington's disease.

In an embodiment, the tRNA Synthetase inhibitor borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, a pharmaceutically acceptable salt thereof and mixtures thereof, among others.

In an embodiment, the invention is directed to a pharmaceutical composition comprising an effective amount of a mixture of at least two tRNA synthetase inhibitors selected from the group consisting of borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839,ganfeborole, PT638, PT662, GSK656, or a pharmaceutically acceptable salt thereof, in combination with a pharmaceutically acceptable carrier, additive and/or excipient.

In an embodiment, the invention is directed to a pharmaceutical composition comprising an effective amount of at least one tRNA synthetase inhibitor selected from the group consisting of borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, or a pharmaceutically acceptable salt thereof, in combination with at least one additional bioactive agent, in combination with a pharmaceutically acceptable carrier, additive and/or excipient.

These and/or other embodiments of the present invention may be readily gleaned from the detailed description of the invention and examples which follow.

### BRIEF DESCRIPTION OF THE FIGURES

FIG. 1 shows that tRNA synthetase inhibitors can increase ATF4 levels in mouse embryonic fibroblasts. A, The ATF4 eGFP translation reporter utilizes ATF4's 5 ′ untranslated region upstream of the GFP start codon [37]. The study design first used the eGFP ATF4 translation reporter to find concentrations of tRNA synthetase inhibitor that upregulate ATF4 translation. B, C ATF4 translation measured in response to varying concentrations of borrelidin (*p<0.05, **p<0.01, ***p<0.001; Dunnett's multiple testing procedure). D, E ATF4 protein levels in response to borrelidin, normalized to total protein level detected by ponceau stain. F, G Phospho-eIF2 and eIF2 protein measured ratio in response to borrelidin, normalized to total protein level detected by ponceau stain (*p<0.05, *p<0.01, ***p<0.001; one-way ANOVA). eGFP=enhanced green fluorescent protein, Veh.=Vehicle, FC=fold change

FIG. 2 shows tRNA synthetase inhibitors upregulate ATF4 activity through Gcn2. A Drugs used in this study and their associated targets. B The amino acid response element measures ATF4 downstream activity. C-I Various tRNA synthetase inhibitors can increase ATF4 downstream activity (*p<0.05, **p<0.01, **p<0.001; Dunnett's multiple testing procedure).

FIG. 3 shows ATF4 causes the differential expression of genes involved with protein turnover. A RNA Seq block design to control for borrelidin treatment and Atf4 knockout sequencing. B Principal component analysis of the 46 samples sequenced. C HOMER motif enrichment from the differentially expressed genes associated with ATF4 (adj. p-value<1e-10). D ClueGo enriched biological process ontology categories (adj. p-value<0.01) of differentially expressed genes (adj. p-value<1e-10) from the linear model result (Design=~ATF4 +Condition). E Heatmap and hierarchical clustering of linear model results (p adj<1e-10). Biological process gene ontology enrichment utilized Panther's ontology resource. F, G Volcano plots of genes differentially expressed from linear model. Both regulation of autophagy (in orange) and proteasomal protein catabolic process (in purple) were found to be enriched in the genes upregulated (p adj<1e-10) with ATF4 (Panther's ontology resource, adj. p-value<0.01). “Up with increased ATF 4” and “Down with increased ATF4” are labeled on either side of the volcano plots presented in association with the limma linear model fit to ATF4 activity given by the linear model study design (Design=~ATF4 +Genotype).

FIG. 4 shows tRNA synthetase inhibitors and ATF4 can decrease protein synthesis in MEFs. A Representative images and histograms of green fluorescence, representing protein synthesis, of wild type and ATF4 KO MEFs treated with various tRNA synthetase inhibitors. Green: Alexa Fluor.™ 488. Blue: NuclearMask™ Blue Stain. B Relative protein synthesis in wild type and ATF4 KO MEFs, normalized to wild type vehicle control in all cases. Dots represent different experiments. (*p<0.01; **p<0.01, ***p<0.001; Students't-test).

FIG. 5 shows tRNA synthetase inhibitors increase caspase-like activity through Atf4. A Peptide and protein aggregation of wild type and ATF4 KO MEFs. Protein aggregation assessed using PROTEOSTAT® divided by the total protein in the sample, normalized to wild type. B Promega's assay schema to measure caspase-like activity of the proteasome. C-I Caspase-like activity increases in response to different tRNA synthetase inhibitors at ATF4-inducing concentrations (*p<0.05, *p<0.01, ***p<0.001; one-way ANOVA).

FIG. 6 shows tRNA synthetase inhibitors cause the activation of the integrated stress response. Proteasome activity and autophagy play a role in the negative feedback loop of amino acid response pathway activation.

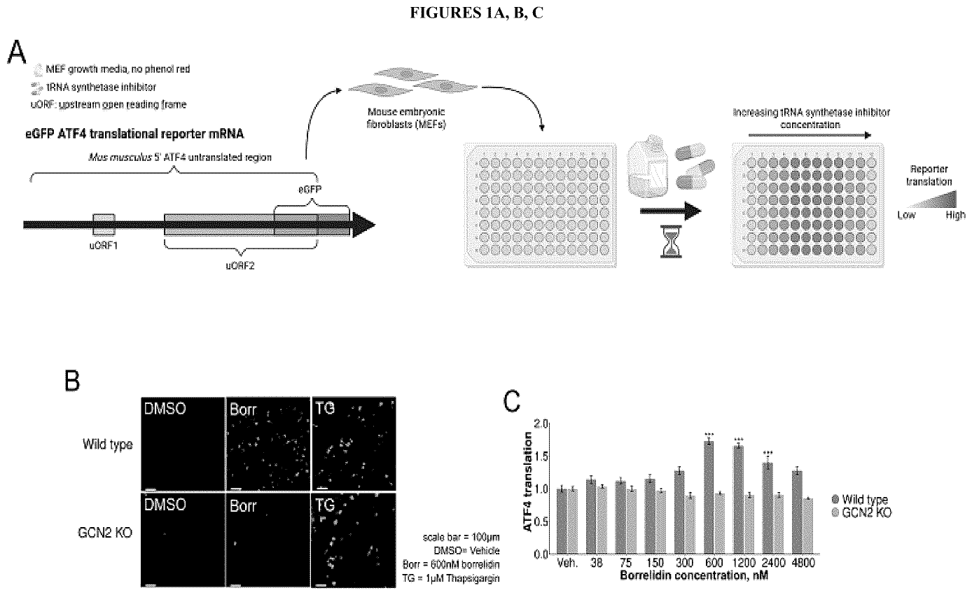

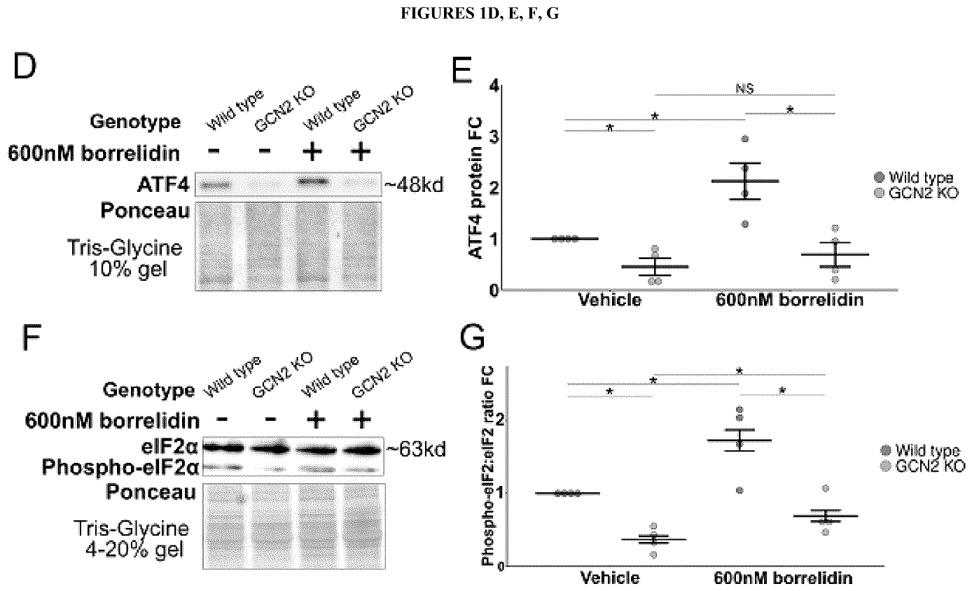

FIG. 1S shows that tRNA synthetase inhibitors increase ATF4 translation through Gcn 2. A) LIVE/DEAD cell viability assay analysis after 24 hours of treatment with varying borrelidin concentration in wild type MEFs. B) ATF4 translation measured in wild type and GCN2 KO MEFs in response to varying borrelidin concentrations at different time points (*p<0.05, **p<0.01, ***p<0.001; linear model: Fluorescence ~Dose+time). C, D) ATF4 protein levels in response to various tRNA synthetase inhibitors. E) eIF2 and Phospho-eIF2 protein levels in response to 600 nM borrelidin. F) 5 and 7 hour incubation times measuring ATF4 translation with 600 nM borrelidin, 10 nM halofuginone, 40 μM tavaborole, 15 μM LysRS-IN-2, 1.5 mM mupirocin, 40 μM REP8839, 60 μM REP3132, and 1 μM thapsigargin. Data is normalized to the hour 5 mean (*p<0.05, **p<0.01, ***p<0.001; Student's t-test). G) 7 hours of treatment of tRNA synthetase inhibitors increase ATF4 translation in a Gcn 2-dependent manner. (*p<0.05, **p<0.01, ***p<0.001; Student's t-test).

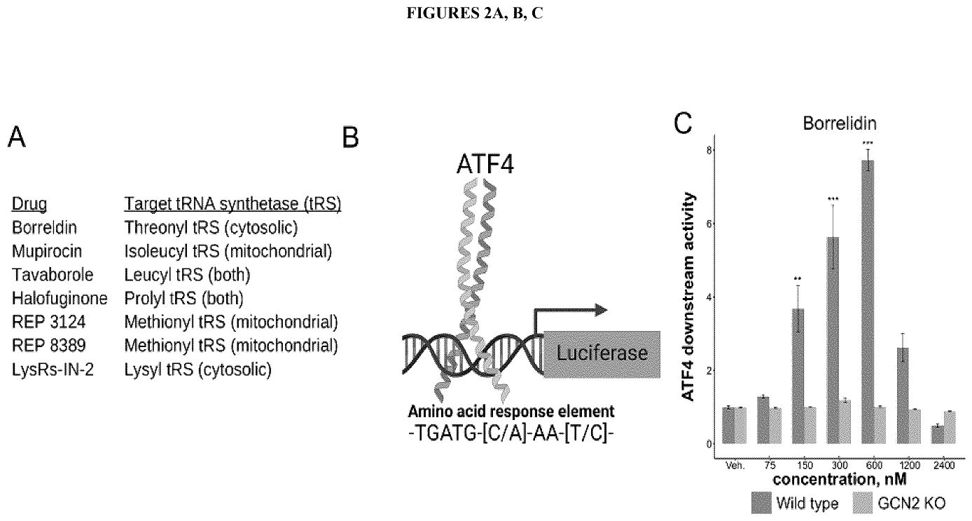

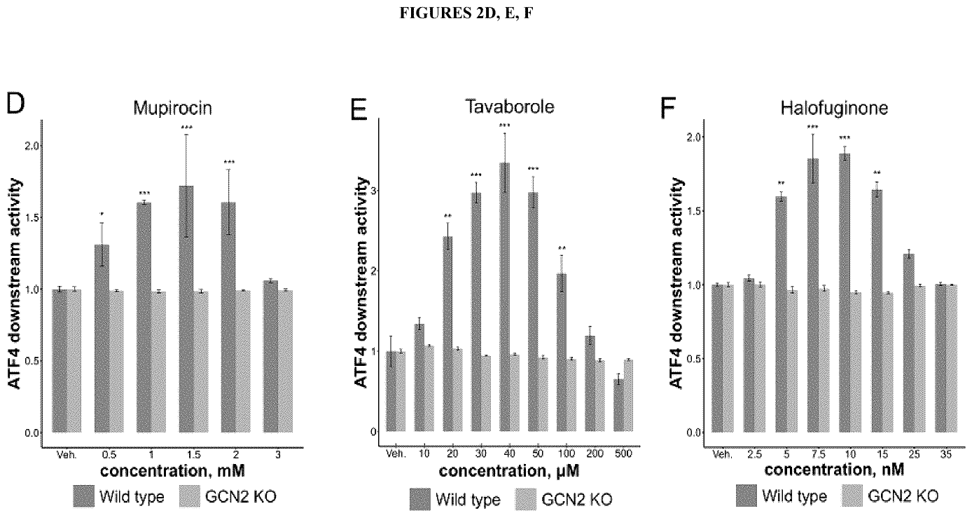

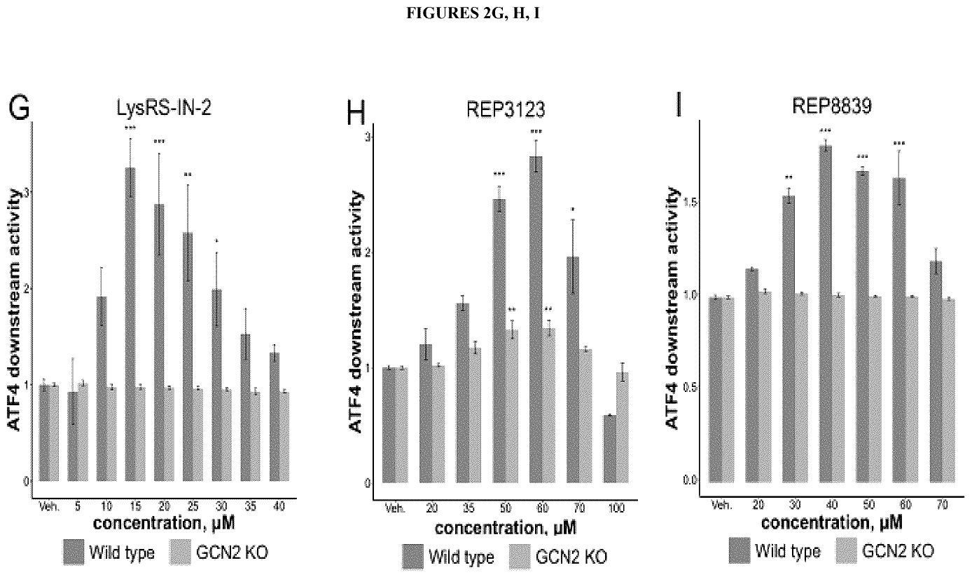

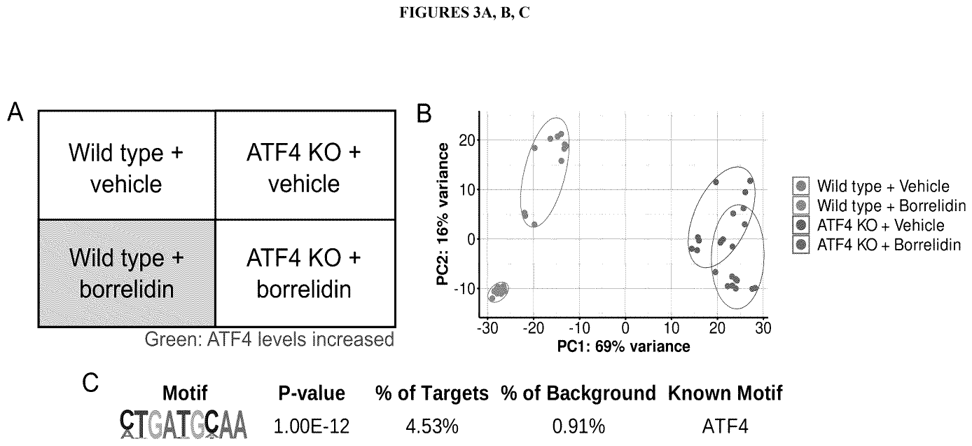

FIG. 2S shows the ATF4 KO MEF cell line generated using CRISPR-Cas9. A) CRISPR-Cas9 targeting ATF4 was transfected and single cells, containing the nuclease were sorted. B) The single cells were grown up and PCR-confirmed. C) The PCR-confirmed samples were sequenced, and an early stop codon was introduced into the Atf4 gene.

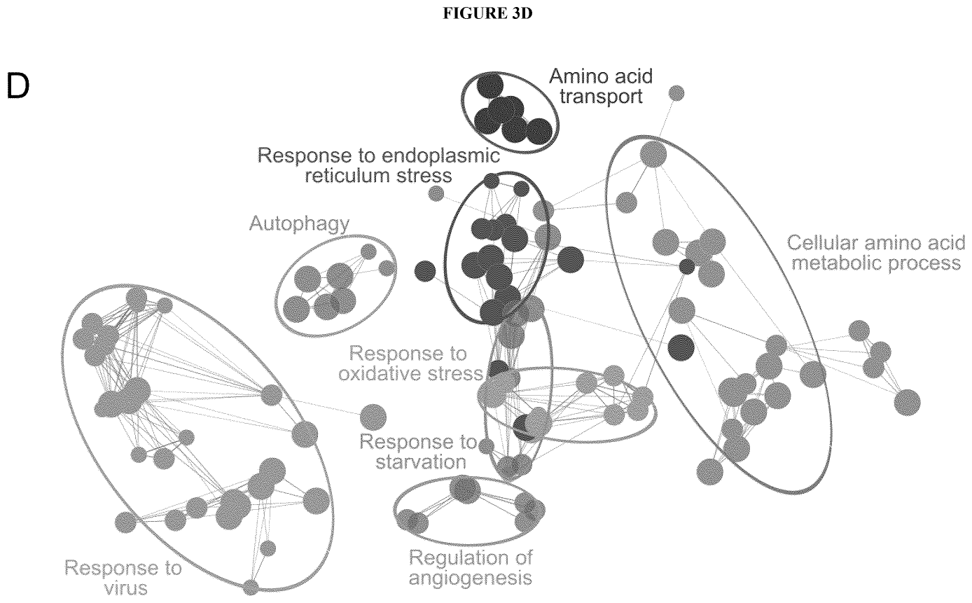

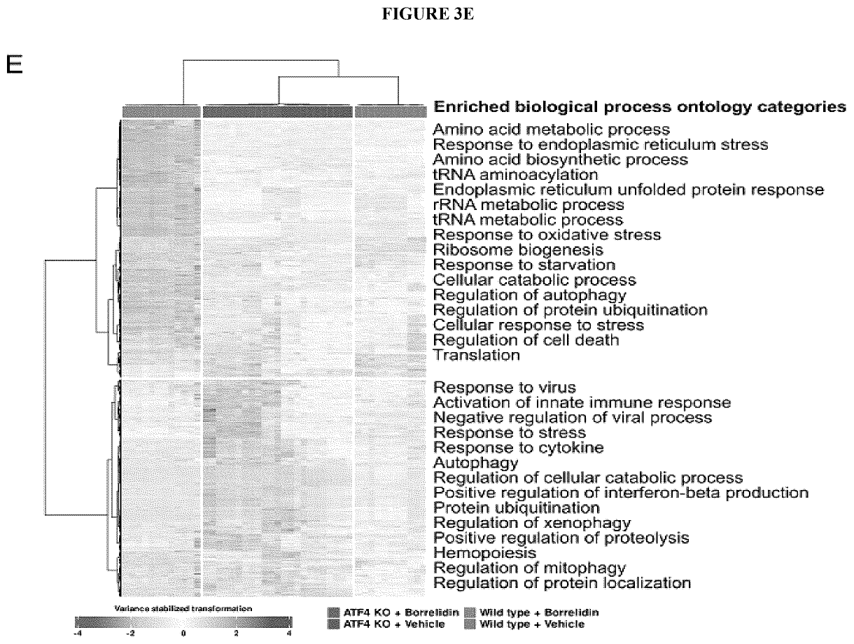

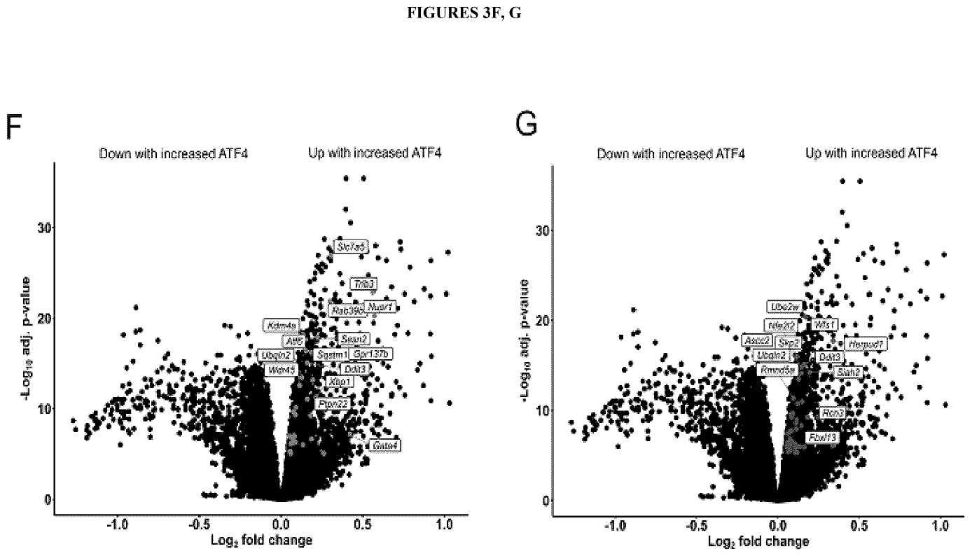

FIG. 3S shows A) qPCR of autophagy genes in MEFs with and without 600 nM borrelidin. B) Enriched molecular function ontology catories from the linear model. C, D) Representative volcano plots of the tRNA synthetase genes differentially expressed from the RNASeq. B Wild type+borrelidin vs. Wild type+vehicle. C) Wild type+borrelidin vs. ATF4 KO+borrelidin. C_{T}=cycle threshold. FDR=false discovery rate.

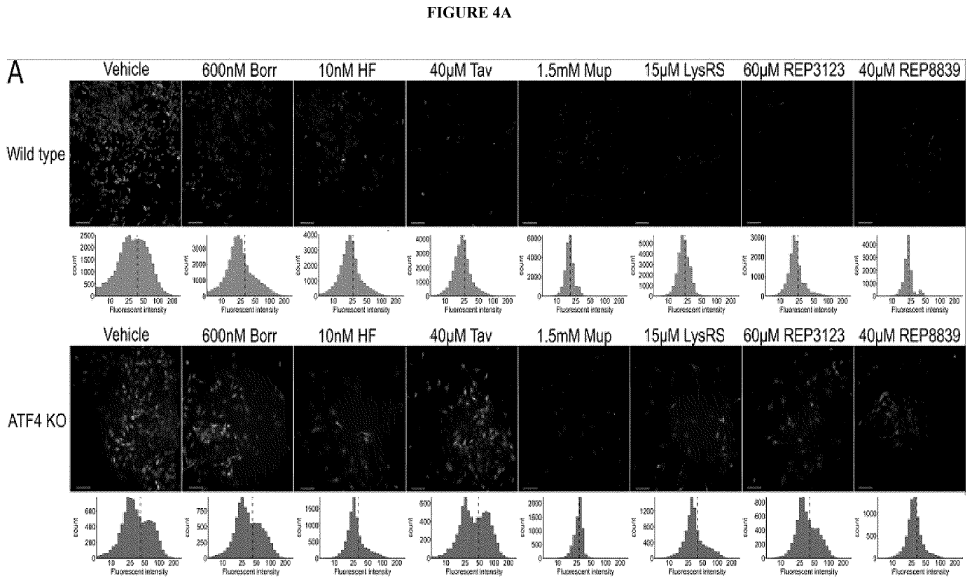

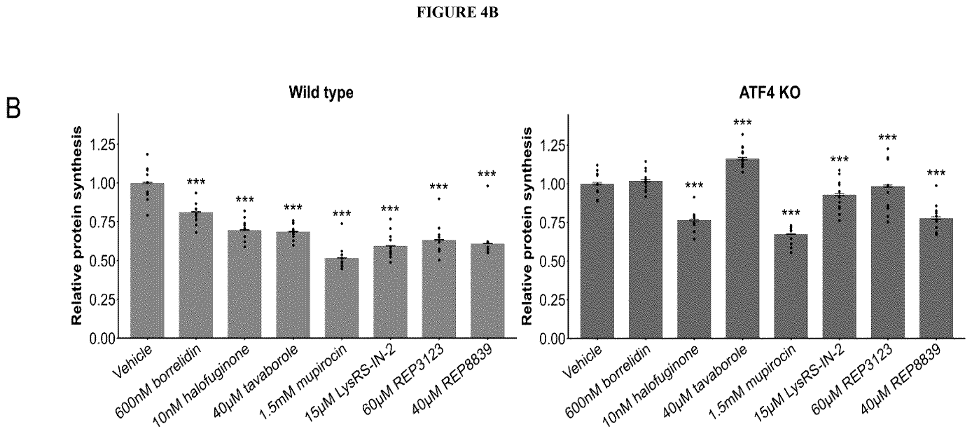

FIG. 4S A-H) shows that Various tRNA synthetase inhibitors increase chemotrypsin-like activity through ATF4 in mouse embryonic fibroblasts. (*p<0.05, **p<0.01, ***p<0.001; one-way ANOVA). MG132=proteasomal inhibitor.

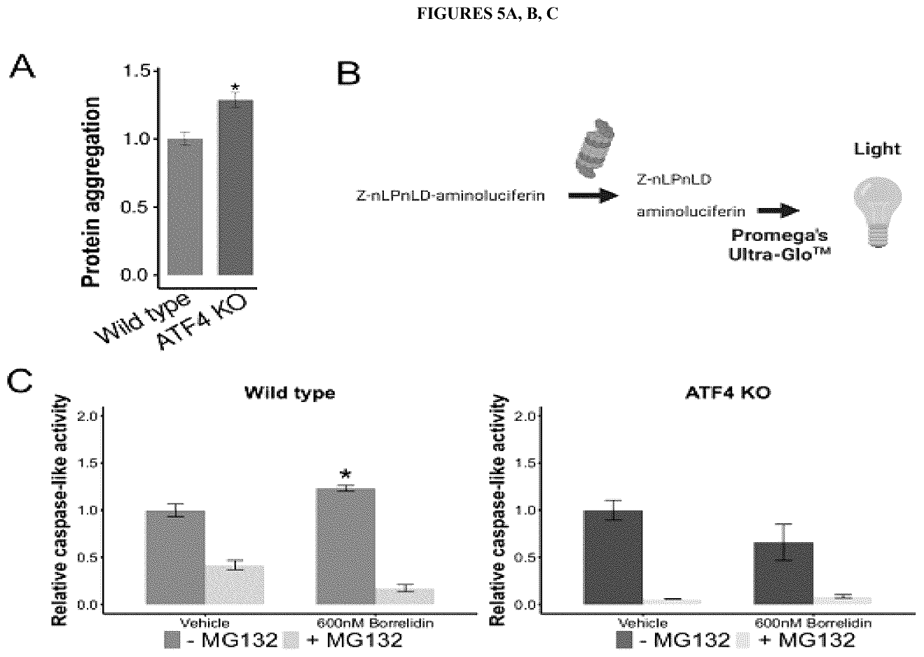

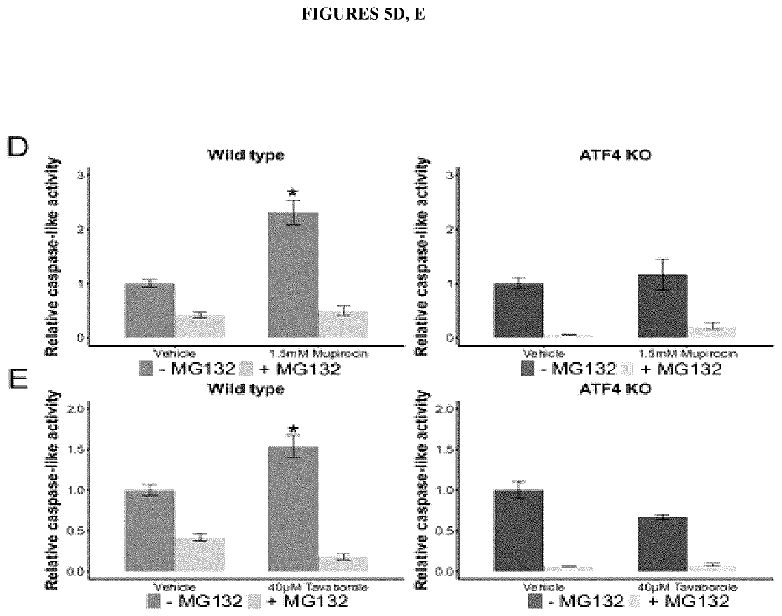

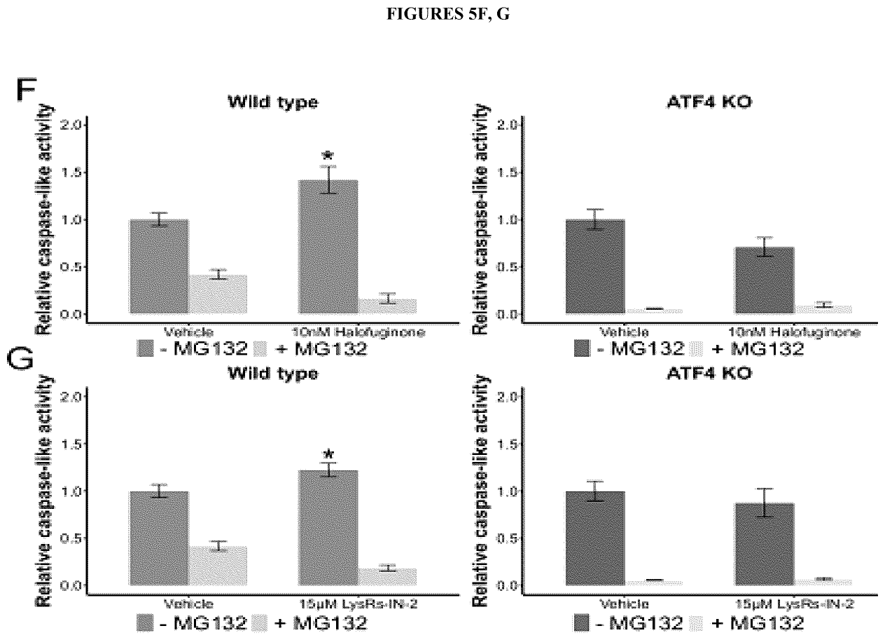

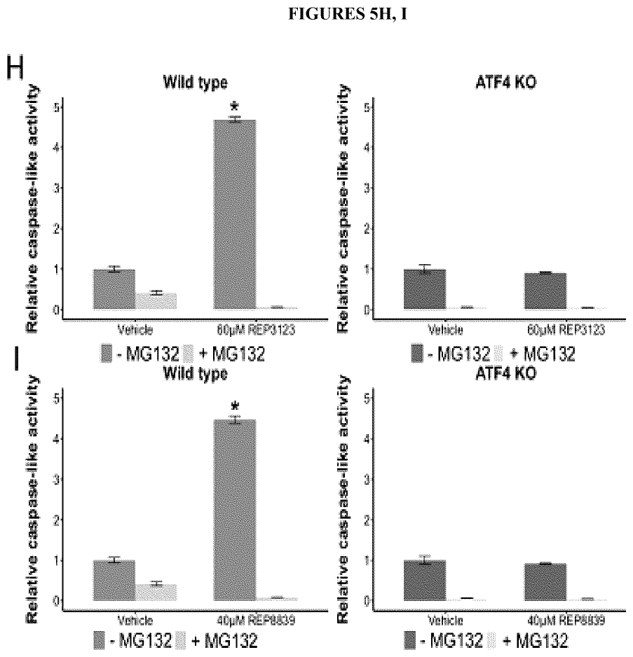

FIG. 5S shows: A) Schema of the LC3ΔG autophagy reporter plasmid. B) Autophagic flux increases in response to tRNA synthetase inhibitors, dependent on Atf4. C) Schema for the ptfLC3 autophagy reporter. D, E, F) Autophagic flux is increased in response to tRNA synthetase inhibitors, dependent on Atf4. G, H) Western blot and its quantification of LC3 autophagy protein in response to tRNA synthetase inhibitors, normalized to total protein by Ponceau stain (*p<0.05, **p<0.01, ***p<0.001; one-way ANOVA).

### DETAILED DESCRIPTION OF THE INVENTION

The following terms shall be used throughout the specification to describe the present invention. A term, which is otherwise not defined, has the same meaning as one of ordinary skill within the context of the use of that term would assign to the term. Note that all terms are used in context to avoid overlap and redundancy where applicable.

The singular forms “a,” “an,” and “the,” include plural referents unless expressly and unequivocally limited to one referent. Thus, for example, reference to “an inhibitor” can include two or more different compounds. As used herein, the term “include” and its grammatical variants are intended to be non-limiting, such that recitation of items in a list is not to the exclusion of other like items that can be substituted or other items that can be added to the listed items.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The term “patient” or “subject” refers to an animal, preferably a mammal, and even more preferably a human, in need of treatment or therapy for pain, especially chronic pain to which compounds according to the present invention are administered in order to treat that condition.

The term “about” when used before a numerical designation, e.g., temperature, time, amount, concentration, and such other, including a range, indicates approximations which may vary by (+) or (−) 20%, 10%, 5% or 1%, or any subrange or subvalue there between. Preferably, the term “about” when used with regard to a dose amount means that the dose may vary by +/−20%.

“Administration” refers to introducing an agent, such as the tRNA Synthetase inhibitor into a patient. Typically, an effective amount is administered, which amount can be determined by the treating physician or the like. Dosages for tRNA synthetase inhibitors, alone or in combination, which are used in the present invention are presented herein below. Any route of administration, such as oral, topical, buccal, subcutaneous, peritoneal, intra-arterial, intravenous, intrathecal, inhalation, vaginal, rectal, nasal, introduction into the cerebrospinal fluid, or instillation into body compartments can be used. The agent, such as a tRNA synthetase inhibitor alone or in combination with additional bioactive agents, may be administered by direct blood stream delivery, e.g. intravenous, sublingual, buccal, intranasal, or intrapulmonary administration.

The term “coadministration” shall mean that at least two compounds or compositions (i.e. a tRNA Synthetase inhibitor in combination with another tRNA synthetase inhibitor or other bioactive agent) are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds according to the present invention may be administered with one or more additional bioactive agents, especially including an additional bioactive treating neurodegenerative diseases as described herein.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of an agent, such as, a tRNA synthetase inhibitor or a pharmaceutically acceptable salt one, two, three, or more times per day. Administration may be via transdermal patch, gum, lozenge, sublingual tablet, intranasal, intrapulmonary, oral administration, intramuscularly or other administration.

The term “comprising” or “comprises” is used to indicate that the compositions and methods include the recited elements, but do not exclude other elements not recited. The term “consisting essentially of” when used to define compositions and methods, shall mean excluding other elements of any essential significance to the combination claimed for the stated purpose. Thus, a composition consisting essentially of the elements as defined herein would not exclude other materials or steps that do not materially affect the basic and novel characteristic(s) of the claimed invention. The term “consisting of” shall mean excluding more than trace elements of other ingredients and substantial method steps. Embodiments defined by each of these transition terms are within the scope of this invention.

The term “effective” is used herein, unless otherwise indicated, to describe an amount of a compound or component which, when used within the context of its use, produces or effects an intended result, whether that result relates to the prophylaxis and/or therapy of an infection and/or disease state, especially a bacterial infection including a infection within the context of its use or as otherwise described herein. The term effective subsumes all other effective amount or effective concentration terms (including the term “therapeutically effective”) which are otherwise described or used in the present application.

The term “therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of a tRNA Synthetase inhibitor, in the context of treating, inhibiting, delaying or reducing the likelihood of a neurodegenerative disease in a patient, refers to an amount of such inhibitor which impacts the patient's neurodegenerative disease within the context of its use.

The term “effective” is used to describe an amount of a compound or component which is included in a composition or in a method of treatment to effect its intended result, whether that result is the treatment, inhibition, delay or reduction in the likelihood of neurodegenerative disease in the patient of need or at risk for disease.

The term “compound”, as used herein, unless otherwise indicated, refers to any specific chemical compound disclosed herein regardless of the treatment or the mechanism by which such effect occurs and includes in context, tautomers, regioisomers (especially cis/trans), geometric isomers, and where applicable, optical isomers thereof, as well as pharmaceutically acceptable salts, solvates and polymorphs thereof. Within its use in context, the term compound generally refers to a single compound and its pharmaceutically acceptable salts, but may also include other compounds such as stereoisomers, regioisomers and/or optical isomers (including in some instances, racemic mixtures) as well as specific enantiomers or enantiomerically enriched mixtures of disclosed compounds, depending on the context of the use of the term. The compounds of this invention include all stereoisomers where relevant (e.g., cis and trans isomers) and all optical isomers of the present compounds (eg., R and S enantiomers), as well as racemic, diastereomeric and/or other mixtures of such isomers, as well as all pharmaceutically acceptable salt forms, solvates, polymorphs and prodrug forms of the present compounds, where applicable.

The term “treat”, “treating” or “treatment” is used to describe the treatment, inhibition or delay of a neurodegenerative disease in a subject or patient pursuant to the administration of compounds or compositions according to the present invention. These terms are therapeutic terms. In the case of prophylaxis, prevention and/or reduction in the likelihood of a neurodegenerative disease occurring in a patient or subject at risk can also be effected.

The term “tRNA synthetase inhibitor” is used to describe compounds and their various pharmaceutically acceptable salts which inhibit Aminoacyl-tRNA synthetases (ARSs). These compounds inhibit these ARS enzymes that ligate amino acids to tRNAs and translate the genetic code during protein synthesis. Aminoacyl-tRNA synthetases (ARSs) are a family of 20 essential enzymes that ligate amino acids to their corresponding tRNAs and translate the genetic code during protein synthesis. The two-step catalytic reaction of ARSs involves the formation of an enzyme-bound aminoacyl-adenylate (AMP-aa) followed by the formation of an aminoacylated tRNA (tRNA-aa) by transferring the amino acid to the corresponding tRNA. This catalytic reaction of ARSs plays a pivotal role in protein synthesis, which is essential for the growth and survival of all cells. Among tRNA synthetase inhibitors for use in the present invention are the compounds borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, and pharmaceutically acceptable salts thereof. These compounds may be used alone in combination with other tRNA synthetase inhibitors or other bioactive agents.

The term “neurodegenerative disease” is used to describe diseases or conditions that gradually cause damage and destroy parts of the nervous system, especially areas of the brain. These conditions usually develop slowly, and the effects and symptoms tend to appear later in life. The term doesn't refer to a single type of condition, but rather to a group of general conditions that applies to several specific types of conditions. These include dementia-type diseases, including Alzheimer's disease, frontotemporal dementia, chronic traumatic encephalopathy (CTE), Lewy body dementia, and limbic predominant age-related TDP-43 encephalopathy (LATE); demyelinating diseases, which involve myelin loss or damage which affects the sending and relaying of nerve signals and include multiple sclerosis (MS), multiple system atrophy and neuromyletis optica spectrum disorder (NMOSD); Parkinsonism-type diseases, which includes Parkinson's disease and other forms of parkinsonism including Huntington's disease; motor neuron diseases such as amyotrophic lateral sclerosis (ALS) and progressive supranuclear palsy (PSP); tauopathies including Pick's disease and corticobasal degeneration and prion diseases which are a type of protein misfolding disease that causes series brain damage in a relatively short period of time and includes Creutzfeldt-Jacob disease (CJD), variant Creutzfeldt-Jacob disease (VCJD), variably protease-sensitive prionopathy (VPSPr), Gerstmann-Sträussler-Scheinker disease (GSS), Kuru and Fatal insomnia (FI).

The term “coadministration” or “combination therapy” is used to describe a therapy in which at least two active compounds in effective amounts are used to treat a disease state or condition as otherwise described herein at the same time. Although the term coadministration preferably includes the administration of two active compounds to the patient at the same time, it is not necessary that the compounds be administered to the patient at the same time, although effective amounts of the individual compounds will be present in the patient at the same time. A tRNA synthetase inhibitor (which term includes any one or more of the compounds or their pharmaceutically acceptable salts, solvates or polymorphs identified herein) according to the present invention may be administered with one or more agents which are traditionally used in the treatment, inhibition or prevention of neurodegenerative diseases. These tRNA synthetase inhibitors include include, for example, borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, and pharmaceutically acceptable salts thereof, among others as described herein. In embodiments, more than on one tRNA synthetic inhibitor may be coadministered to a patient in need or at risk.

Thus, the present invention is directed to a method for treating, inhibiting, ameliorating, delaying or reducing the likelihood of a neurodegenerative disease or condition, including dementia-type diseases, demylenating diseases, parkinsonism-type diseases, motor neuron diseases, taupathies and prior diseases. These diseases include Alzheimer's disease, frontotemporal dementia, chronic traumatic encephalopathy (CTE), Lewy body dementia, and limbic predominant age-related TDP-43 encephalopathy (LATE), multiple sclerosis (MS), multiple system atrophy and neuromyletis optica spectrum disorder (NMOSD), Parkinson's disease, Huntington's disease, amyotrophic lateral sclerosis (ALS), progressive supranuclear palsy (PSP), Pick disease, corticobasal degeneration, Creutzfeldt Jacobs disease (CJD), variant Creutzfeldt Jacobs disease (VCJD), variably protease-sensitive prinopathy (VPSPr), Gerstmann-Sträussler-Scheinker disease (GSS), Kuru and Fatal insomnia (FI), the method comprising administering an effective amount of a tRNA synthetase inhibitor to the patient in need of treatment, including a patient at risk of a neurodegenerative disease state or condition.

The present invention is also directed to pharmaceutical compositions which consist essentially of an effective amount of effective amounts of at least two tRNA synthetase inhibitors as described herein or a tRNA synthetase inhibitor in combination with an additional bioactive agents, generally an agent which is traditionally used in the treatment of a neurodegenerative disease or condition such as aducanumab, solanezumab, semorinemab, verubecestate, semagacesta (for AD), prasinezumab, affitope PD01a, a mGluR5 antagonist, a mGlur agonist, a mGlur4 agonist, SV2C and LRRK2 (for PD), or a pharmaceutically acceptable salt thereof, optionally in combination with an effective amount of a pharmaceutically acceptable carrier, additive or excipient.

The present invention also includes the compositions comprising the pharmaceutically acceptable salt. i.e., the acid or base addition salts of compounds of the present invention and their derivatives. The acids which may be used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others. In a preferred aspect the dihydrochloride salt of pramiprexole (pramiprexole dihydrochloride or Mirapex) is used as the active pramiprexole agent.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines, among others.

Regardless of the mechanism, the compounds of the present invention may be used to treat disease states or conditions in patients or subjects who suffer from those conditions or disease states or are at risk for those conditions. In this method a compound in an effective amount is administered to a patient in need of therapy to treat, inhibit, delay, ameliorate and/or reduce the likelihood of a neurodegenerative disease or condition in a patient in need. The concentration of compound to be administered to a patient in need will vary depending on the nature of the compound, the route of administration, the pharmacokinetics of the compound, etc., but effective amounts of compound often will fall within the range of 0.001 mg/kg to 50 mg per kg, often 0.01 mg/kg to 25 mg/kg or 0.05 mg/kg to 10 mg/kg or 0.1 mg to 5 mg/kg by weight of the patient to be treated.

Compositions according to the present invention may be administered by any conventional means known in the art. Pharmaceutical formulations include those suitable for oral, rectal, nasal, topical (including buccal and sub-lingual), vaginal or parenteral (including intramuscular, sub-cutaneous, intrathecal and intravenous) administration. Compositions according to the present invention may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. When desired, the above-described formulations may be adapted to provide sustained release characteristics of the active ingredient(s) in the composition using standard methods well-known in the art.

In the pharmaceutical aspect according to the present invention, the compound(s) according to the present invention is formulated preferably in admixture with a pharmaceutically acceptable carrier, additive or excipient. In general, it is preferable to administer the pharmaceutical composition orally, but certain formulations may be preferably administered parenterally and in particular, in intravenous, intrathecal or intramuscular dosage form, as well as via other parenteral routes, such as transdermal, buccal, subcutaneous, suppository or other route, including via inhalation intranasally. Oral dosage forms are preferably administered in tablet or capsule (preferably, hard or soft gelatin) form. Intravenous, intrathecal and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity.

Compositions suitable for parenteral injection may comprise physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coatings or shells, such as enteric coatings and others well known in the art. They may also contain opacifying agents, and can also be of such composition that they release the active compound or compounds in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, sesame seed oil, glycerol, tetrahydrofurfuryl alcohol, polyethylene glycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Suspensions, in addition to the active compound, may contain suspending agents, as for example, ethoxylated isostearyl alcohols, polyoxyethylene sorbitol or sorbitan esters, microcrystalline cellulose, aluminum metahydroxide, bentonite, agar-agar, or tragacanth, or mixtures of these substances, and the like.

Compositions for rectal or vaginal administration, where applicable, can be prepared by mixing an active agent and any additional compounds with suitable non-irritating excipients or carriers such as cocoa butter, polyethylene glycol or a suppository wax, which are solid at ordinary room temperature, but liquid at body temperature, and therefore, melt in the rectum or vaginal cavity and release the active.

Dosage forms for topical administration include ointments, powders, sprays and inhalants. The compound(s) are admixed under sterile conditions with a physiologically acceptable carrier, and any preservatives, buffers, and/or propellants that may be required. Opthalmic formulations, eye ointments, powders, and solutions are also contemplated as being within the scope of this invention.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and the type of neurodegenerative disease and/or condition to be treated, and the particular mode of administration. In embodiments, the active agent(s) is used in effective amounts. Preferably, the compositions should be formulated to contain between about 0.01 milligram to about 500-750 milligrams or more, more preferably about 0.05 milligram to about 50 milligrams, and even more preferably about 0.1 milligrams to about 25 mg of a tRNA synthetase inhibitor and optionally, at least 0.01 milligrams to about 500-750 milligrams or more, about 0.05 mg to about 50 milligrams and about 0.1 mg to about 25 mg of at one additional least one additional active agent as otherwise described herein.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular pain condition being treated.

### EXAMPLES

### Methods

### Mouse Embryonic Fibroblast Cell Culturing and ATF4 KO Cell Line Generation

Wild type and GCN2 KO mouse embryonic fibroblast (MEFs) were purchased from American Type Culture Collection global biological resource center. The ATF4 KO cell line was made in house using utilizing optimized guide RNAs optimized from the available

CRISPR/Cas9 genome editing tools with Integrated DNA Technologies targeting the Atf4 gene. Transfection of the assembled Cas9 complex utilized the NEON electroporation system by ThermoFisher and fluorescence-activated cell sorting with the sy3200 Cell Sorter from Sony Biotechnology in collaborations with the UNM Comprehensive Cancer Center Support Grant NCI P30CA118100 and the UNMHSC Flow Cytometry shared resource (Supplementary FIG. 2). All cell culture procedures were done in a BSL-2 laminar flow cabinet with cells of a low progeny. All sub-culturing procedures utilized guidance from peerpublished resources, without Penicillin-Streptomycin [64, 65]. All plasmid transfections utilized Viafect (Promega) following the manufacturer instructions. Halofuginone was purchased from Ambeed [CAS No. 64924-67-0]. Borrelidin was purchased from BioViotica (BVT-0098). Thapsigargin was purchased from AdipoGen (AG-CN2-0003). Bafilomycin A1 was purchased from Sigma-Aldrich (19-148). LysRS-IN-2 was purchased from GLPBio (GC65058). REP3123 and REP8839 were purchased from Axon Medchem (1704, 1705). Mupirocin was purchased from BOC Sciences (B0084-056590). MG-132 was purchased from Sigma-Aldrich (M7449). Tavaborole was purchased from Cayman Chemical (23101).

Fluorescence and luciferase assays in MEFs ATF4 translation experiments utilized a fluorescent translational reporter for ATF 4: ATF 4 5:5′ATF4.GFP was a gift from David Ron (Addgene plasmid #21852; website: n2t.net/addgene: 21852; RRID: Addgene_21852) [36].ATF4 translation over time was measured with the BioTek Synergy HTX Multi-Mode Microplate Reader shared resource in the UNMHSC Autophagy, Inflammation, and Metabolism Center for Biomedical Research Excellence (AIM) core. The AIM core is supported by NIH grant P20GM121176 from NIGMS. All luciferase assays utilized a Victor NIVO multimode plate reader. ATF4 downstream activity was measured using the pGL4[luc2P/ATF4-RE/ Hygro] Vector, which was purchased from Promega utilizing a previously well-understood ATF4 binding element [66, 67]. Proteasomal assays utilized (1) Promega's Proteasome-Glo Caspase-like Cell-Based Assay (G8660) and (2) Abcam's Proteasomal Activity Kit (ab107921) according to the manufacturers'procedures respectively [68, 69]. Protein aggregation assays utilized Enzo Life Sciences'PROTEOSTAT® (51023) according to the manufacturers'protocols.

All LIVE/DEAD, autophagy, and protein synthesis assays were quantified by the Cellinsight CX7 high-content screening platform by ThermoFisher in the UNMHSC AIM core. LIVE/DEAD assay utilized the ATT Bioquest Live or Dead Cell Viability Assay Kit (Cat. No. 22789). Autophagy assays utilized two autophagic flux reporters. pMRX-IP-GFP-LC3-RFP-LC3ΔG was a gift from Noboru Mizushima (Addgene plasmid #84572; website: n2t.net/addgene: 84572; RRID: Addgene_84572) and ptfLC3 was a gift from Tamotsu Yoshimori (Addgene plasmid #21074; website: n2t.net/addgene: 21074;RRID: Addgene_21074) [70, 71]. Widefield microscopy to supplement the findings utilized the UNMCCC Fluorescence Microscopy and Cell Imaging Shared Resource which is supported by University of New Mexico Comprehensive Cancer Center Support Grant NCI P30CA118100. Protein synthesis assays were conducted with the Click-iT™ HPG Alexa Fluor™ 488 Protein Synthesis Assay Kit (ThermoFisher Cat. No. C10428) following the manufacturer's protocol, where fluorescence surrounding the cell nucleus was quantified in a high-throughput manner, representing the rate of protein synthesis for each cell.

### MEF RNA-Seq and Analysis

RNA was extracted from ~70% confluent MEFs after 7 h of condition exposure in accordance with the block design in 60-mm cell culture dishes. DMSO of 1% v/v was used for the controls and 600 nM borrelidin delivered in DMSO at congruently 1% v/v was used in accordance with the high downstream ATF4 transcriptional activity evidenced from the ATF4 downstream luciferase assays (FIGS. 2E, 2F). RNA was extracted using Zymo's RNA extraction kit according to the manufacturer's protocols. A total of 48 samples were sent to GeneWiz and all except two passed RNA quality control tests, resulting in n ≥11 for each condition. After read quality control and fastp processing, fastq files were aligned and quantified utilizing HISAT2 and feature counts [72-75]. Data analysis and differential expression utilized R and the libraries limma, for differential expression analysis and creation of the linear model, and DESEQ2, for data visualization and differential expression analysis [76, 77].

### Western Blotting Analysis of MEF Proteins

Cell protein extraction and Western blot analyses were done using standard procedures. Briefly, protein samples were extracted using RIPA Lysis and Extraction Buffer (Genesee Sci. Cat. No. 18-415) using manufacturer's protocol followed by suspension in Laemmli Sample Buffer (Bio-rad Cat. No. #1610737EDU) and heating to 98° C. for 5 min to denature the proteins. Protein was quantified and loaded equally into electrophoresis gels using Pierce™ BCA Protein Assay Kit (ThermoFisher Cat. No. 23225). The protein samples were loaded into Novex™ 4 to 20% or 10% Tris-Glycine Plus, 1.0 mm, Midi Protein Gels (Invitrogen Cat. No. WXP42012BOXA) in Tri/Glycine/SDS buffer. After electrophoresis, the gel was transferred to an Immun-Blot® PVDF Membrane (Bio-rad Cat. No. #1620175) in Tris/glycine transfer buffer with 10% methanol. Membranes were blocked in 5% dried milk in TBST with tween. The membranes were immunoblotted with the following primary antibodies: LC3 (Sigma, Cat. No. L8918), ATF4 (Proteintech Cat. No. 10835-1-AP), eIF 2α (ThermoFisher Cat. No. PA 5-41916), Phopho-eIF 2α (ThermoFisher Cat. No. 44-728G). 10 μg/well of protein was loaded for ATF4 and LC 3 western blots while 20 μg/well was loaded for eIF 2α and Phospho-eIF 2α blots. The secondary antibody used was an appropriate horseradish peroxidase-conjugated antibody, in which the membranes were incubated in for 1 h at room temperature. The antibody-antigen complex was visualized by a Clarity™ Western ECL Substrate (Bio-rad Cat. No. 170-5061). The intensities of the bands were quantified using the Gel Imager program, by normalizing the band intensity of proteins of interest to the lane's total protein quantified by Ponceau [78].

### Statistical Analysis

Statistical significance of ATF4 fluorescent and luciferase assays was calculated using the Dunnett's multiple comparison procedure versus the vehicle. Statistical significance for all autophagy fluorescence assays were calculated using the Dunnett's multiple comparison procedure versus the vehicle. Statistical significance of all western blots were calculated using a one-way ANOVA. All data displaying the changes in ATF4 levels, translation, or transcriptional reporter activity are normalized to the vehicle control, representing fold change (FC) with error bars representing the standard error of the normalized fold change mean. Unless specified otherwise, all error bars are standard error of the mean. Any noted statistical significance is reported in the figure legend. Significantly differentially expressed genes were found from the limma R package, using the Kenward-Roger approximation for linear models. Over-represented ontologies were calculated using both Panther and ClueGo using Benjamini-Hochberg multiple testing correction for False Discovery Rate calculation. All caspase-like and chemotrypsin-like assay p-values were calculated utilizing a one-way ANOVA. All caspase-like and chemotrypsin-like assay data presented is presented as fold change (FC) in relation to the vehicle control. All statistics and display of data was done with R programming.

### Results

Multiple tRNA synthetase inhibitors can increase ATF4 in mammalian cells

ATF4 is regulated translationally by two upstream open reading frames (uORFs) in its 5′ untranslated region, and a delay in translation re-initiation results in increased ATF4 translation [36, 37]. We first measured ATF4 translation in response to tRNA synthetase inhibitors using an eGFP-ATF 4 translational reporter containing the 5′ untranslated region of ATF4 fused to eGFP in place of ATF4 in mouse embryonic fibroblasts (MEFs) (FIG. 1A) [36]. We first sought to ask if borrelidin, a threonyl tRNA synthetase inhibitor, could increase ATF4 translation through the GCN2-mediated amino acid response leg of the integrated stress response [79, 80]. To find the therapeutic window in culture, we first assessed cell viability in an increasing doses of drug and found that 24 h of 2 mM borrelidin treatment in media almost completely killed the cells (Supplemental FIG. 1SA). From there, we dialed in the treatment incubation time over a dose range of 0 to 4.8 mM and found that 7 h was consistently sufficient to upregulate ATF4 translation at doses from 150 to 2400 nM (Supplemental FIG. 1SB). After that, we found that this ATF4upregulation was dependent on Gcn2, utilizing thapsigargin (TG), an inducer of the ISR through the PKR-like endoplasmic reticulum kinase, as a positive control (FIG. 1B, C, Supplemental FIG. 1SB). We confirmed this ATF4 upregulation with western blots (FIGS. 1D, E, Supplemental FIGS. 1SC, 1SD). To further test if tRNA synthetase inhibitors are acting through the AAR, we assessed the ratio of phosphorylated-eIF2α to eIF2α with western blots (FIGS. 1F, G, Supplemental FIG. 1SE). We found both wild type and GCN2 KO MEFs had a significant rise in eIF2α phosphorylation levels in relation to its vehicle counterpart, however the GCN2 KO cells, however, they were not significantly higher than the wild type vehicle condition. Together, these data suggest that borrelidin acts through the ISR, specifically through the phosphorylation of eIF2α by the GCN2 uncharged tRNA sensor, in order to increase ATF4 translation.

The inventors next tested six additional inhibitors targeting different tRNA synthetases (FIG. 2A). We found that a 7-h incubation time with these drugs was sufficient to increase ATF4 translation in wild type MEFs, similar to what we saw with borrelidin (Supplemental FIG. 1SF). We next utilized an amino acid response element luciferase reporter where luciferase is only transcribed when ATF4 binds to its known consensus binding sequence (FIG. 2B) [66, 67, 81]. All of these inhibitors showed a bell-shaped response curve of ATF4 downstream activity with inhibitor dose, after 7 h of treatment (FIG. 2C-I). At lower doses, activation increases with increasing drug concentration via GCN2-mediated increased translation of ATF4. At higher doses, these drugs likely inhibit overall translation enough to offset the specific induction of ATF4.Borrelidin was the most potent ATF4 activity inducer, increasing its activity by more than sevenfold at 600 nM after 7 h of treatment (FIG. 2C). Mupirocin, despite targeting the mitochondrial isoleucyl tRNA synthetase, was similarly found to increase ATF4downstream activity and translation through Gcn2 (FIG. 2D, Supplemental FIG. 1SG) [82, 83]. The antifungal agent tavaborole, a leucyl tRNA synthetase inhibitor, was found to increase ATF4 translation and downstream activity through Gcn2 (FIG. 2E, Supplemental FIG. 1SG) [82]. Previous work utilizing halofuginone, a prolyl tRNA synthetase inhibitor, has been shown to increase ATF4 levels in vitro, and our studies confirm this (FIG. 2F, Supplemental FIG. 1SD) [54]. Lys-RS-IN-2, a lysyl tRNA synthetase inhibitor, was also found to increase ATF4 activity dependent on Gcn2 (FIG. 2G, Supplemental FIG. 1SG) [84]. REP3123 and REP8839, two methionine tRNA synthetase inhibitors known for their potent affinity for the prokaryotic tRNA synthetase enzyme, were similarly found to increase ATF4 activity (FIGS. 2H, I) [85, 86]. For all inhibitors except for REP3123, induction of ATF4 downstream activity was completely dependent on Gcn2. We reasoned that methionine tRNA synthetase inhibitors may be able to induce ATF4 independently of Gcn2, as charged methioninyl-tRNA is a component of the translation pre-initiation complex and thus translation initiation may be delayed when methionine tRNA synthetase activity is impacted [34, 87]. Taken together, these data suggest that ATF4 translation and downstream activity can be upregulated in response to multiple inhibitors of different tRNA synthetases in mammalian cells.

ATF4 Upregulation Causes the Differential Expression of Genes Associated with Protein Turnover

After we identified highly inducing doses of tRNA synthetase inhibitors in MEFs, we sought to analyze the ATF4-dependent changes in the cell's transcriptome. We knocked out Atf4 using an Integrated DNA Technologies-adapted CRISPR-Cas9 protocol (Supplementary FIG. 2S) and we designed an RNA-sequencing experiment to analyze the ATF4 transcriptional targets under high activity using linear model analysis of a block design with high biological replicates (n≥11 per treatment) (FIG. 3A) [76, 88]. The block design features the ability to simultaneously control for the two variables: (1) tRNA synthetase inhibitor treatment and (2) ATF4 activity levels. We chose to conduct the RNASeq with borrelidin as it was the most potent inducer of the ATF4 transcriptional response. Principal component analysis of the 46 samples revealed that principal component 1 was able to separate the samples by their genotype while principal component 2 separated based on the treatment of drug (FIG. 3B). Principal component 2 separated the wild type samples based on their drug treatment much better than it did the ATF4 KO samples, suggesting that borrelidin impacted the two different cell lines differently, and in line with our expectation that many of the transcriptional changes upon borrelidin treatment might depend on increased ATF4 translation.

After alignment of reads and quantification, genes with changed mRNA levels associated with ATF4 activity were pulled out based on the linear model fit to increased ATF4 levels (Interactive volcano plot of results provided in File 1) [76]. FIGS. 3 C-F present the findings of this linear model design (Design=~ATF4 +Genotype), which upon fitting, results in the log fold change and adjusted p-values of transcripts in association with the documented ATF4 activity from FIG. 2. Using HOMER motif analysis, we confirmed that the immediately upstream presumptive promoter sequences of genes whose transcript levels changed with ATF4 activity (padj<1e-5) showed enrichment of the known ATF4 amino acid response binding element [A/G]-TT-[G/T]-CATCA (p<1e-12) (FIG. 3C) [66, 67, 89]. The enriched biological process ontology categories from the genes differentially expressed from the linear model fit (padj<1e-10) were plotted on an edge-node graph utilizing ClueGo (FIG. 3D) [90].

We next used hierarchical clustering of the genes found to be significantly (padj<1e-10) associated with linear model fit (FIG. 3E). Encouragingly, the samples (columns) clustered cleanly in alignment with their genotype and drug treatment. This clustering further revealed that genes associated with autophagy, protein catabolism, translation, and other stress responsive genes are differentially expressed downstream of ATF4. We also analyzed the enriched biological process gene ontology categories of the genes upregulated in conditions of high ATF4 activity, and found many processes involved in protein degradation were over-represented (regulation of autophagy (GO: 0010506), proteasomal protein catabolic process (GO: 0010498), ERAD pathway (GO: 0036503)) (FIGS. 3F, G, interactive volcano plot included in File 1). Further, these data present differentially expressed genes known to impact protein synthesis, by the amino acid biosynthesis ontology categories shown to be enriched here, in alignment with what is already known about Atf4 and its orthologs [16, 91]. Altogether, these data indicate that ATF4 may be a key regulator of protein turnover, changing the expression of genes involved in protein synthesis and protein degradation.

Many novel genes were found to be significantly differentially expressed with ATF4 activity, including Wipi2, a gene essential in the LC3 lipidation step of autophagy, which was upregulated with high ATF4 activity [92, 93]. Quantitative PCR of autophagy genes Atg2 a, Atg7, Atp13a2, and Wipi2 further confirmed higher levels of the associated genes'mRNA in borrelidin-treated wild type samples, in agreement with our RNASeq results for these genes (p<0.05) (Supplemental FIG. 3SA). Our RNASeq results also showed increased expression of a significant number of genes encoding tRNA synthetases when ATF4 is induced, including Tars, Gars, Cars, Nars, Yars, Sars, Lars, Wars, Mars1, Vars, and Eprs (padj<1e-3), consistent with other studies (Supplemental FIG. 3SB, 3SC, 3SD) [52,94]. Conceptually, these data suggest that ATF 4 increases the expression of these tRNA synthetases to correct for the scenario that the ISR was activated by the accumulation of uncharged tRNAs, potentially due to a malfunctioning tRNA synthetase.

tRNA Synthetase Inhibitors and ATF4 can Decrease Protein Synthesis

From the RNASeq results, there were many genes found to be differentially expressed with increased ATF4 activity having to do with translation, indicating that ATF4impacts protein synthesis, aligning with other studies [16, 91, 95, 96]. As protein turnover mechanisms are tightly controlled by the balance between protein synthesis and degradation, we sought to understand the impact of our drugs and ATF4 on protein synthesis. We found that wild type cells treated with every different tRNA synthetase inhibitor at doses that increase ATF4 levels had significantly reduced translation (FIG. 4A). We next assessed the impact of tRNA synthetase inhibitors on ATF4 KO cells and found that 5/7 tRNA synthetase inhibitors had reduced translation, while borrelidin and tavaborole either had no significant change, or increased protein synthesis. Consistently, wild type cells have lower protein synthesis than ATF4 KO cells when treated with the same dosage of drug, implicating ATF4 as a reducer of protein synthesis, in agreement with other studies [16, 91, 95, 96]. However, cells treated with halofuginone, mupirocin, LysRS-IN-2, REP3123, and REP8839 still had significantly reduced protein synthesis in comparison to their vehicle counterpart in both wild type and ATF4 KO cells (FIG. 4B).

### Trna Synthetase Inhibitors Can Upregulate Protein Degradation in an Atf4Dependent Manner

Protein turnover in an organism is balanced by both protein synthesis and protein degradation. This knowledge, along with the results from the RNASeq indicating a change in autophagy and proteasomal activity, led us to next assess peptide and protein aggregation in cell lines with and without Atf4 (FIG. 5A). Intriguingly, the untreated ATF4 KO cell line had significantly more aggregation than the untreated wild type cell line, which could be due to impaired unfolded protein response in ATF4 KO cells [97]. ATF4 is also among the individual proteins degraded by the UPS, suggesting the possible existence of a negative feedback loop [98, 99]. To further investigate ATF 4's influence on protein degradation, we assessed the proteasome's caspase-like activity (FIG. 5B). We found that borrelidin is able to significantly increase proteasomal caspase-like activity in wild type MEFs (FIG. 5C). However, in ATF4 KO MEFs, borrelidin did not increase proteasomal caspase-like activity, suggesting that Atf4 is necessary for increased caspase-like proteasomal activity upon borrelidin treatment. Similar results were found in all of the tested tRNA synthetase inhibitors at concentrations that we found them to induce ATF4 activity (FIGS. 5D-I). Interestingly, the mitochondrial methionine tRNA synthetase inhibitors, REP3124 and REP8839, induced proteasomal caspase-like activity the most, agreeing with previous work on responses to direct methionine restriction [100]. We also assessed the proteasome's chemotrypsin-like activity, and found chymotrypsin-like proteasomal activity to be induced in an Atf4-dependent manner by all 7 inhibitors as well, mirroring our findings with caspase-like proteasomal activity (Supplemental FIGS. 4SA-4SH). Taken together, these data show that tRNA synthetase inhibitors can increase proteasomal activity in mammalian cells in an Atf4-dependent manner.

As genes involved in autophagy were also identified in our RNASeq results (FIGS. 3D-F), we next assessed the change in autophagy in response to tRNA synthetase inhibitors, and found that autophagic flux is upregulated upon tRNA synthetase inhibitor treatment, completely dependent on Atf4. We demonstrated this utilizing three different, independent autophagy assays. The first assay used an autophagic flux GFP-LC3-RFP-(LC3ΔG) fluorescent reporter, and showed that autophagic flux increases in tRNA synthetase inhibitor treated samples, dependent on Atf4 (Supplemental FIGS. 5SA, 5SB). We next utilized the ptfLC3 autophagic flux reporter, which again showed that autophagic flux levels rose in response to tRNA synthetase inhibitors, dependent on Atf4 (Supplemental FIGS. 5SC, 5SD, 5SE, 5SF). Finally, we quantified levels of LC3II by western blot and found that they were increased upon tRNA synthetase inhibitor treatment, again dependent on Atf4(Supplemental FIGS. 5SG, 5SH). LC3II levels are linked to the formation of the autophagosome and increased autophagy [49]. These results further indicate that tRNA synthetase inhibitors can increase autophagy in an Atf4-dependent manner, aligning with other studies [54]. Altogether, these results show tRNA synthetase inhibitors can increase protein degradation pathways through Atf4 in mammals, outlining Atf4 as a key contributor to the regulation of protein turnover.

### Discussion

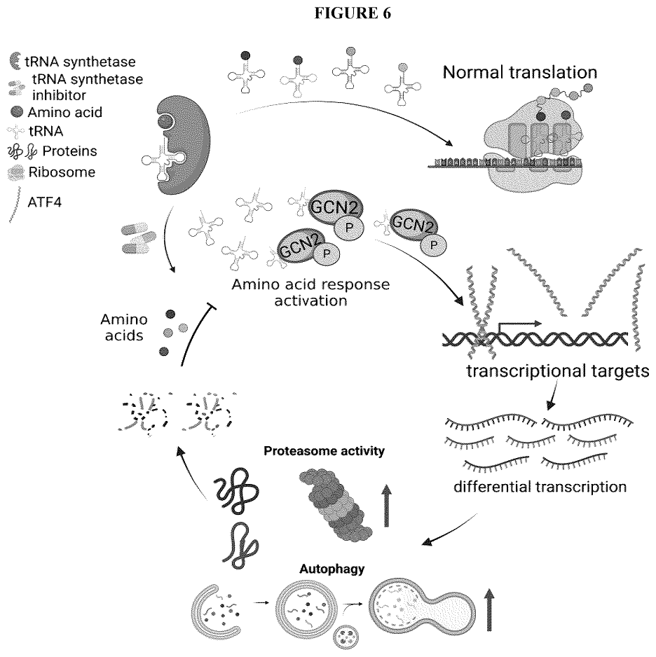

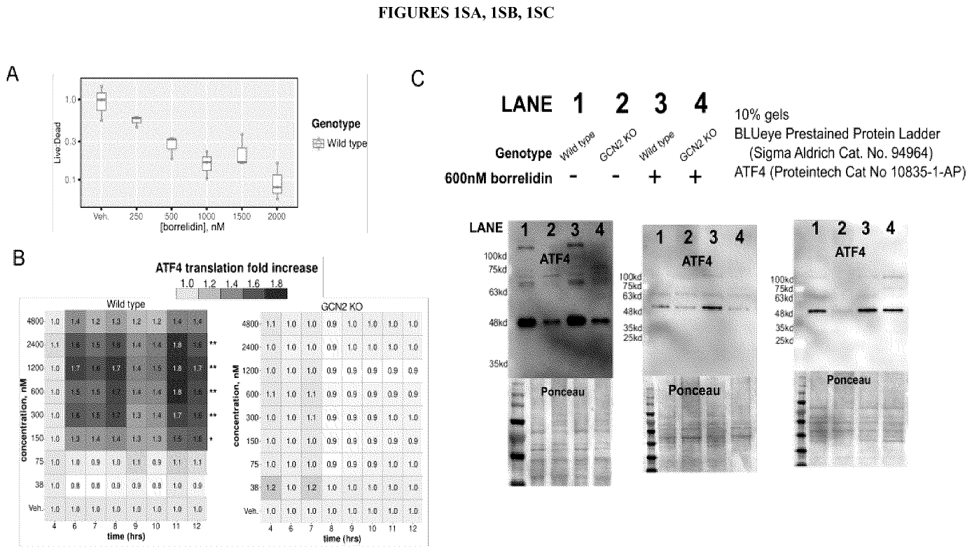

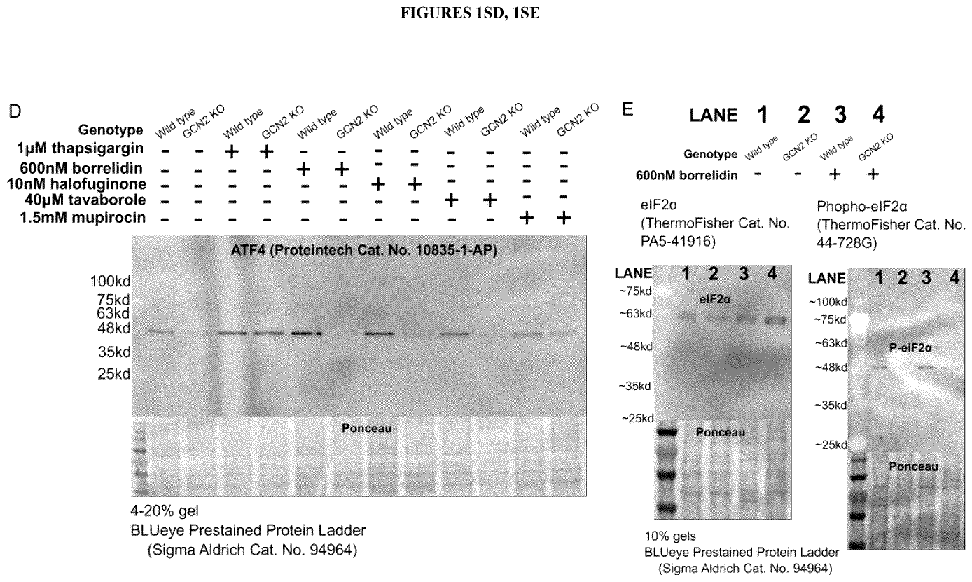

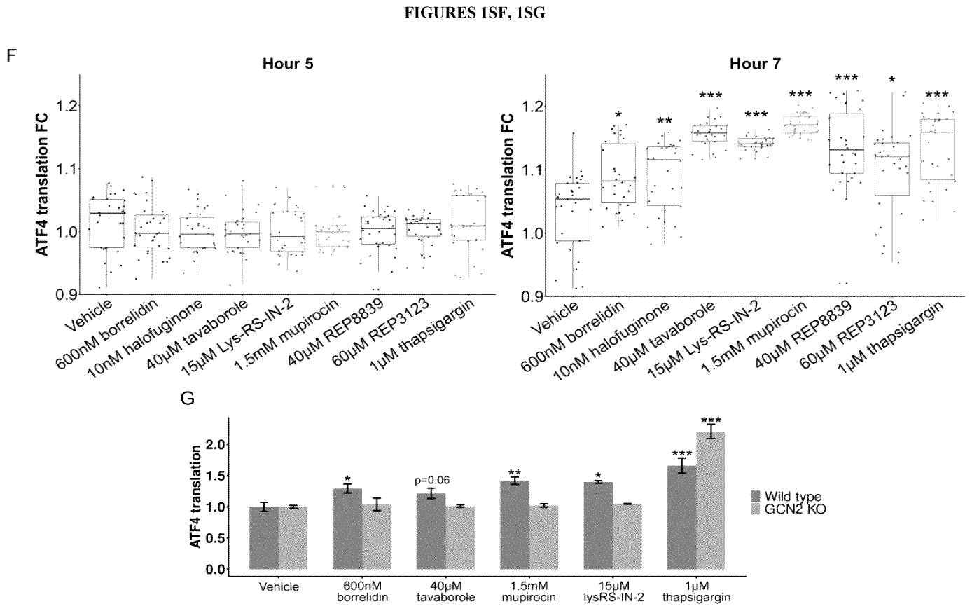

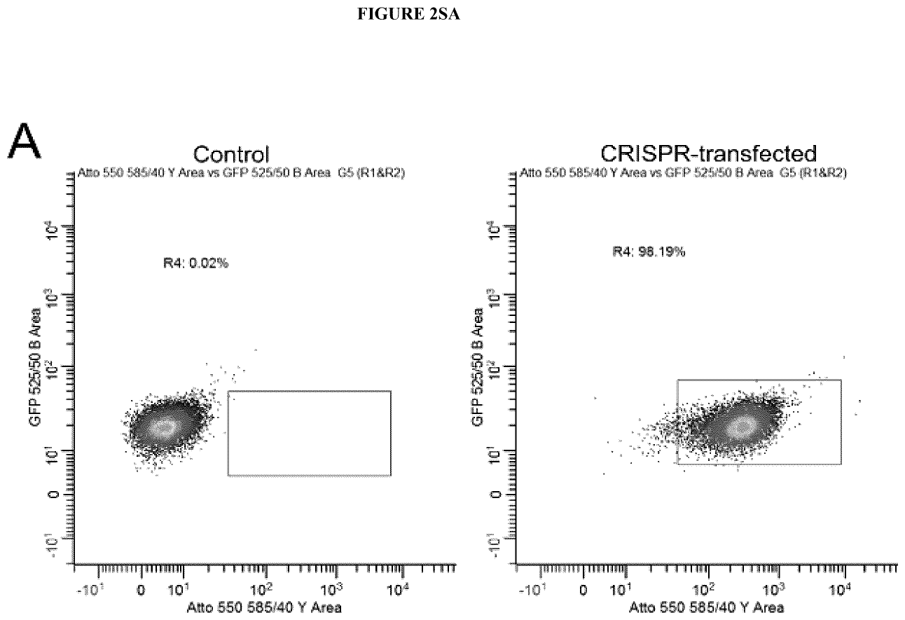

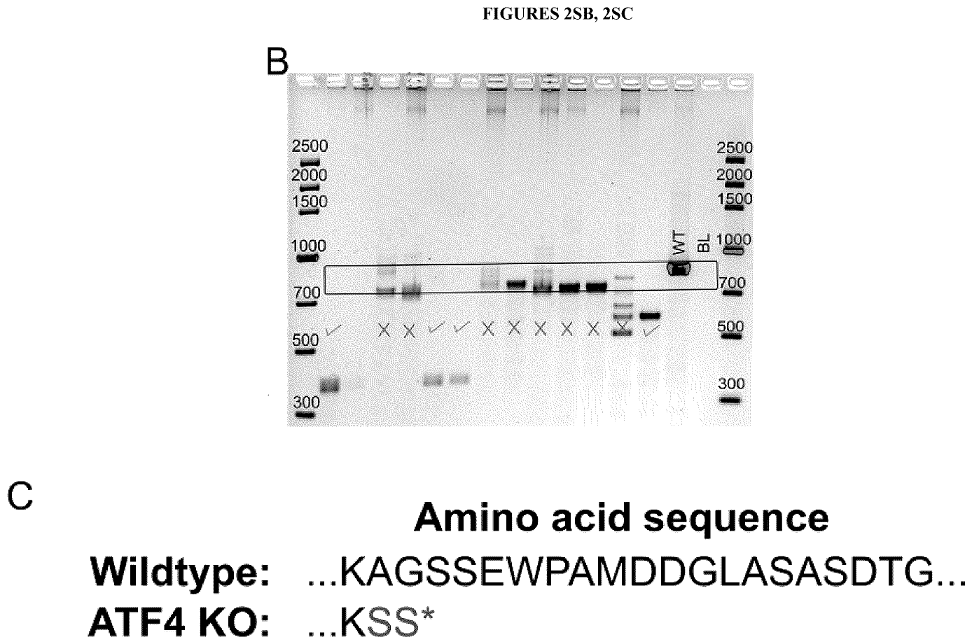

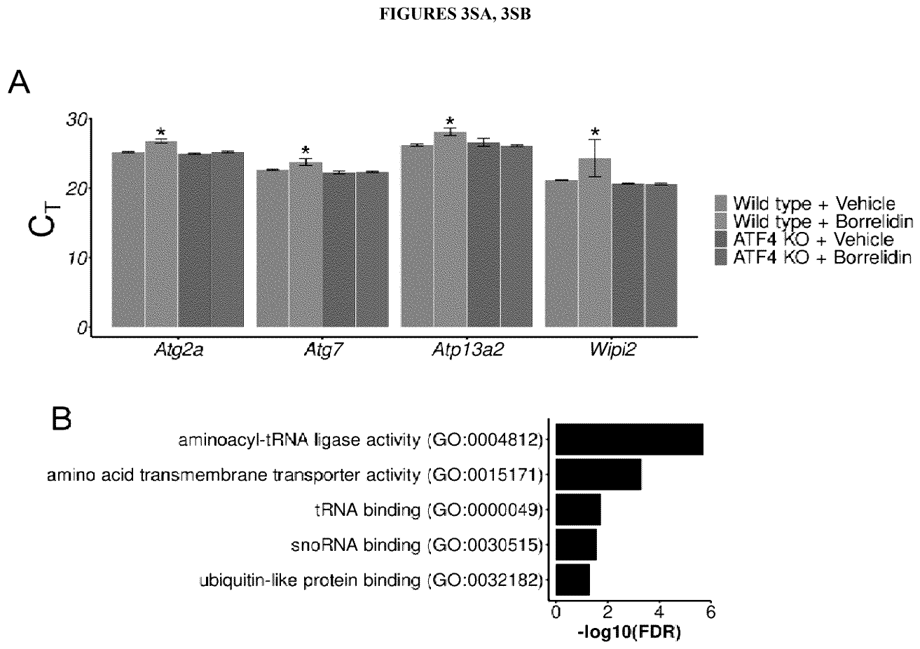

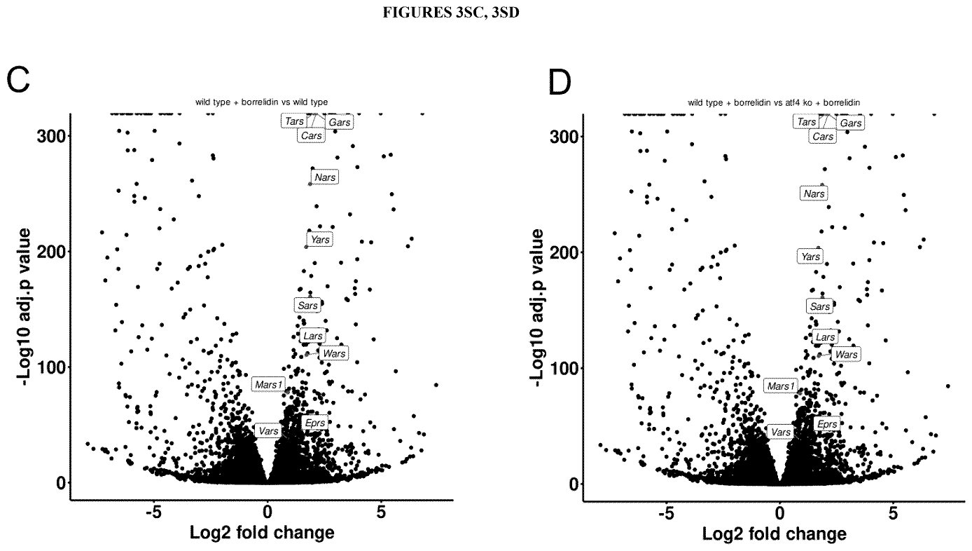

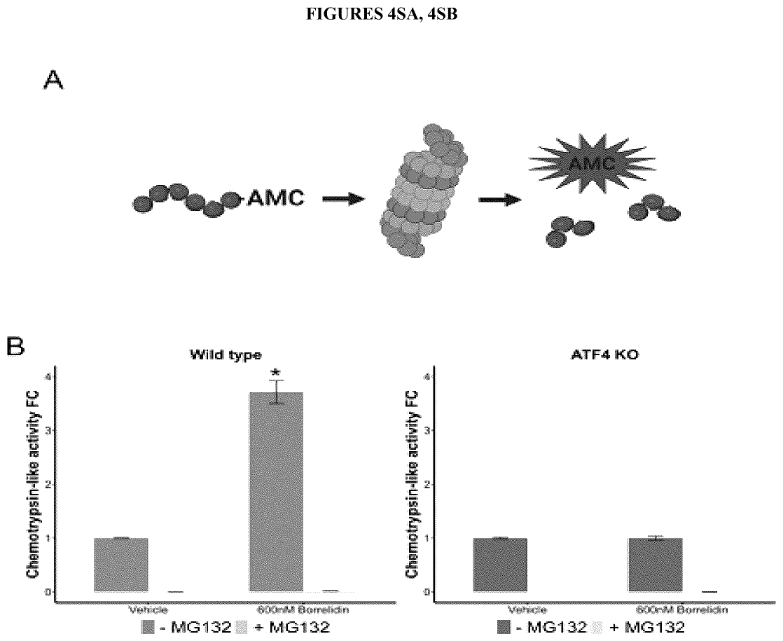

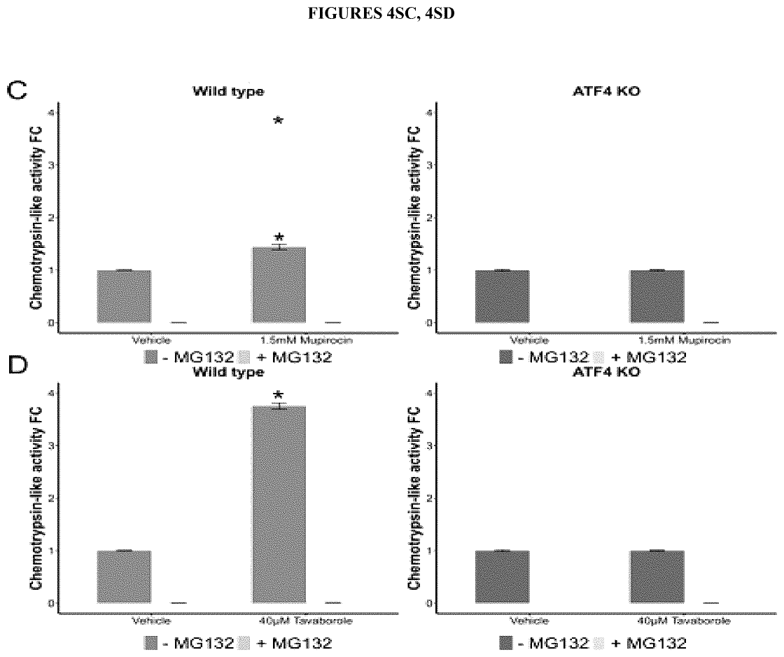

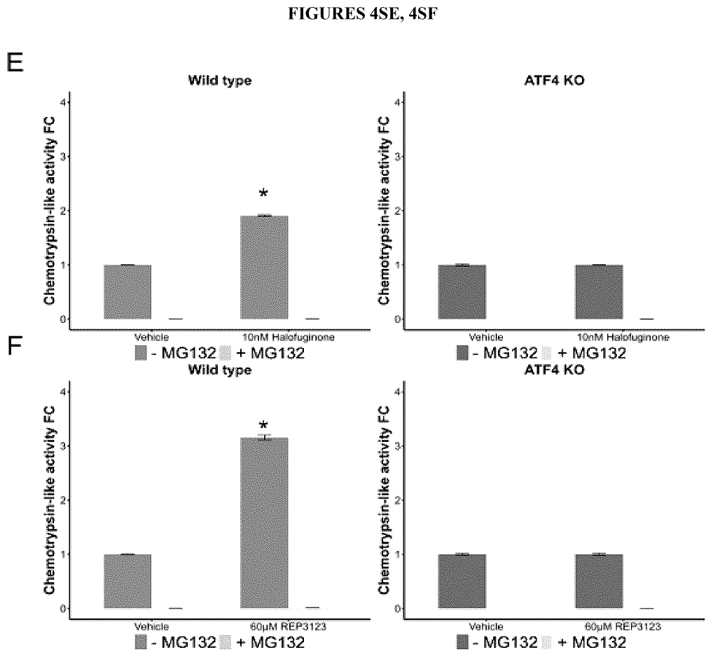

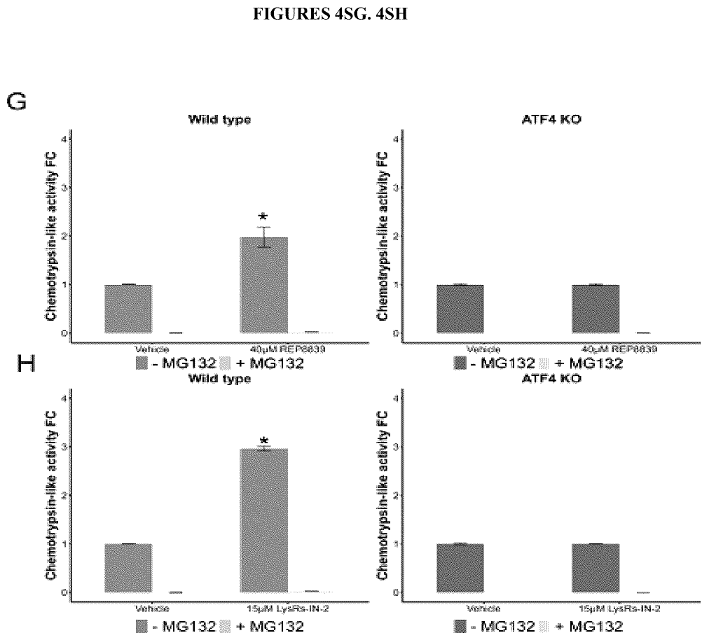

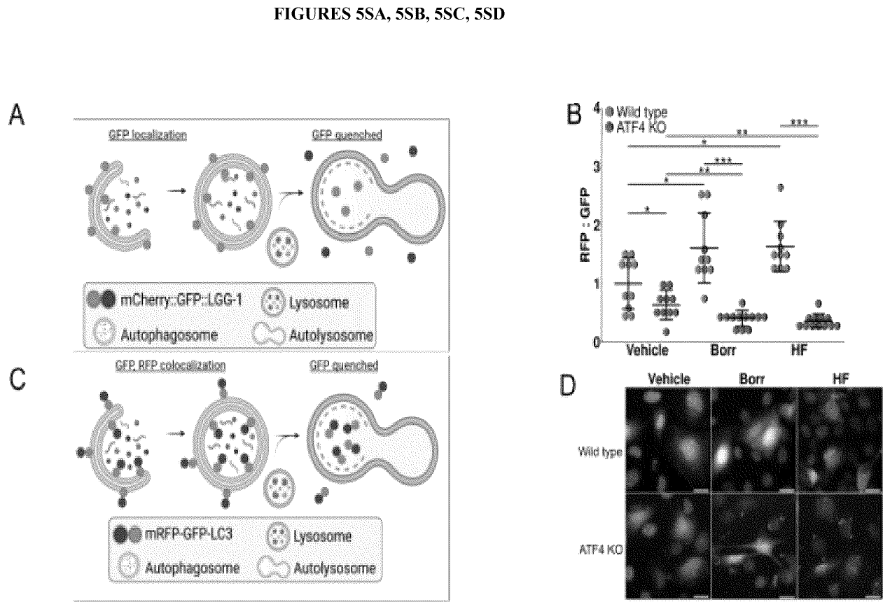

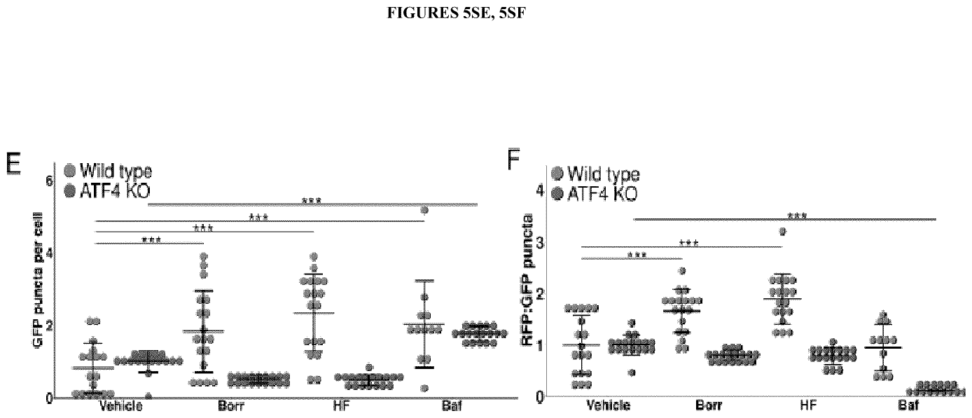

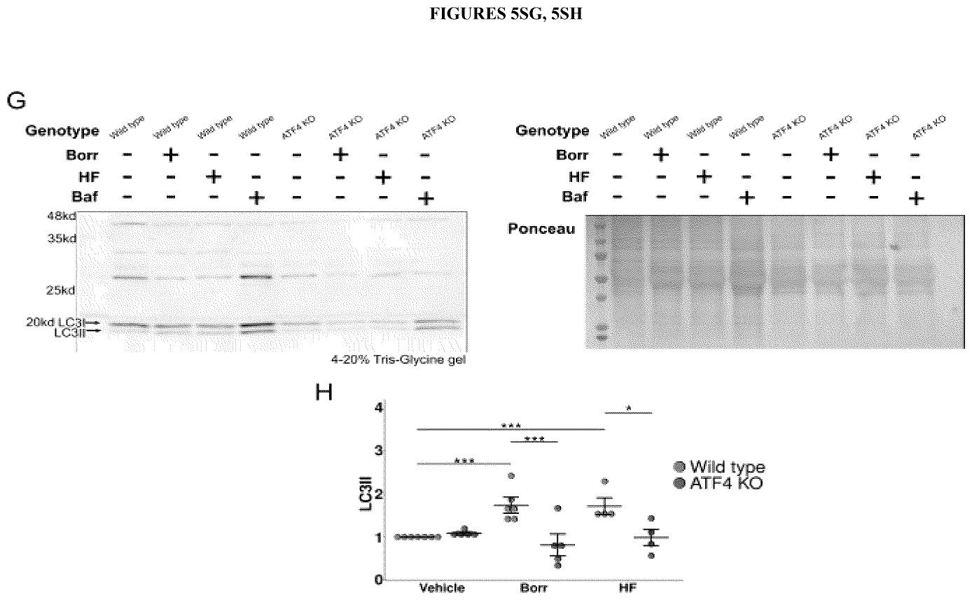

Pharmacological inducers of the UPS are scarce but have the potential to treat many diseases such as Huntington's disease, Alzheimer's disease, and Parkinson's disease [2, 3, 5, 58, 101, 102]. Pharmacological autophagy inducers also have the potential to play roles in protection against neurodegenerative diseases and aging [103-105]. Notably, we found multiple distinct tRNA synthetase inhibitors can dramatically upregulate the UPS in mammalian cells through Atf4 (FIG. 6) [98, 99]. The drugs presented here, including two tRNA synthetase inhibitors that have been shown to increase lifespan in yeast and worms, have major implications for the biological activity of ATF4 and its potential impact on the aging process [27]. Further, the unfolded protein response is a pathway ending with ATF4 that is known to increase lifespan in many model organisms [97, 106-109]. This study, along with the growing literature surrounding ATF4, underscores the highly important role ATF4 potentially plays in many longlived models that may converge on improved proteostasis, itself a hallmark of aging [11-13, 110, 111].

Cells are likely to interpret uncharged tRNAs as a sign of amino acid starvation, regardless of whether the uncharged tRNA stems from the cytoplasm or mitochondria [112]. Given that GCN2 contains a histadyl tRNA synthetase-like domain that is thought to have the ability to recognize many types of uncharged tRNA, it logically follows that GCN2 may be able to sense both cytosolic and mitochondrial uncharged tRNA [16, 19,112-115]. Its activation leads to the translation of ATF4 and likely similar downstream responses no matter the type of uncharged tRNA that initiated this cascade, supporting what we found from testing various tRNA synthetase inhibitors that inhibit both cytosolic and mitochondrial tRNA synthetases. To that end, we found that protein synthesis is decreased and proteasomal activity is increased in wild type cells in comparison to their ATF4 KO counterparts. While our current model is that mupirocin, REP3132, and REP8839 act only as inhibitors of their mitochondrial tRNA synthetases respectively, we have not ruled out the possibility that these compounds act on the cytosolic enzymes as well, or even on other previously undiscovered targets.

In order to further assess these drugs'potential for therapeutic uses, researchers must ensure that the dosing of their organisms leads to the high upregulation of ATF4 or its orthologs. Of note, only certain ranges of concentrations of these drugs greatly increase lifespan in yeast and worms, dependent on their ATF4 orthologs [27]. Underdosing may have little to no effect, and over-dosing may have a detrimental effect, aligning with other studies (and further evidenced here in Supplemental FIG. 1SA) [116].

ATF4 and its orthologs are also known to impact protein synthesis [91, 95, 96, 117]. The results presented here show ATF4 can decrease protein synthesis in response to doses of some inhibitors, but it alone is not responsible for the reduction of protein synthesis in mupirocin, halofuginone, LysRS-IN-2, REP3123, and REP 8839 treated cells. Previous work outlined that borrelidin decreases protein synthesis in both wild type and gcn4Δ yeast; however, replicative lifespan is only increased in the wild type strains [27]. This, along with these data here, indicate that lowered translation is not the sole contributor to lifespan increase and that other protein degradation processes regulated by Gcn4/ATF4, such as autophagy or the UPS, may play a necessary role, warranting further investigation. However, these data uncouple elevated proteasome activity and reduced protein synthesis.

Although this manuscript outlines the argument that ATF4 is beneficial in many contexts of aging, there are contexts in which ATF4 may play a negative role [67, 118, 119]. For instance, recent work by Miller et al. has shown that ATF4 KO mice have maintained muscle mass with age [119]. This may be due to the fact that those mice developed without Atf4, suggesting that development without Atf4 can result in alternative pathways regulating the same phenomenon. Another perspective may be that since the data presented here associates Atf4 with protein degradation mechanisms, perhaps the loss of Atf4 results in impaired protein degradation capacity and thus decreased atrophy. This could explain why the maintenance of muscle mass is improved in the Miller et al. ATF4KO mouse model. Further, ATF4 is considered a cancer target as it has been found to be pro-oncogenic; when upregulated, ATF4 can give cancerous cells the ability to live with limited nutrient delivery, potentially due to its positive relationship with protein degradation mechanisms such as autophagy [52, 53, 120-124].

Conceptually, many of the processes found to be enriched in our RNASeq results may have evolved to be influenced by ATF4 in the event the ISR was activated by amino acid deprivation (i.e. the accumulation of uncharged tRNA) to increase amino acid availability and decrease amino acid usage [16, 20, 113, 125]. Similarly, since the integrated stress response is also activated by endoplasmic reticulum stress and the presence of double-stranded RNA, it follows that ATF4 would logically impact genes that would have the ability to respond these activators of the ISR as well [16]. The RNASeq data here illustrates that even though we increased ATF4 translation through GCN2, we found many differentially expressed genes tied to the biological process categories unfolded protein response, viral presence response, and endoplasmic reticulum stress (FIG. 3). These data further outline the roles ATF4 plays in response to ISR activation, consistent with previous findings, as a multifaceted transcription factor [50-52].

Inducing processes that activate pathways known to promote protein turnover has great interest in the development of therapeutics for diseases characterized by protein aggregation, including important diseases of aging such as Huntington's and Alzheimer's diseases [105]. The results presented here showing that tRNA synthetase inhibitors greatly upregulate proteasomal degradation as well as autophagy open the door for further exploration of tRNA synthetase inhibitors as potential treatments for these very important diseases of aging.

### REFERENCES

1. Coux O, Tanaka K, Goldberg A L. structure and functions of the 20S and 26S proteasomes. Annu Rev Biochem. 1996; 65:801-47. https://doi.org/10.1146/annurev. bi.65.070196.004101.
2. Seo H, Sonntag K-C, Kim W, Cattaneo E, Isacson O. Proteasome activator enhances survival of Huntington's disease neuronal model cells. PLoS One. 2007; 2(2): e238. https://doi.org/10.1371/journal.pone.0000238.
3. Chondrogianni N, Sakellari M, Lefaki M, Papaevgeniou N, Gonos E S. Proteasome activation delays aging in vitro and in vivo. Free Radic Biol Med. 2014; 71:303-20. https://doi.org/10.1016/j.freeradbiomed.2014.03.031.
4. Nguyen N N, Rana A, Goldman C, Moore R, Tai J, Hong Y, Shen J, Walker D W, Hur J H. Proteasome B5 subunit overexpression improves proteostasis during aging and extends lifespan in Drosophila Melanogaster. Sci Rep. 2019; 9(1): 3170. https://doi.org/10.1038/s41598-019-39508-4.
5. Yazgili AS, Ebstein F, Meiners S. The proteasome activator PA200/PSME4: an emerging new player in health and disease. Biomolecules. 2022; 12(8): 1150. https://doi.org/10.3390/biom12081150.
6. Dahlmann B, Becher B, Sobek A, Ehlers C, Kopp F, Kuehn L. in vitro activation of the 20s proteasome. Enzyme Protein. 1993; 47(4-6): 274-84. https://doi.org/10.1159/000468685.
7. Cascio P. PA 28γ: new insights on an ancient proteasome activator. Biomolecules. 2021; 11(2): 228. https://doi.org/10.3390/biom 11020228.
8. Kruegel U, Robison B, Dange T, Kahlert G, Delaney J R, Kotireddy S, Tsuchiya M, Tsuchiyama S, Murakami C J, Schleit J, Sutphin G, Carr D, Tar K, Dittmar G, Kaeberlein M, Kennedy B K, Schmidt M. Elevated proteasome capacity extends replicative lifespan in Saccharomyces Cerevisiae. PLoS Genet. 2011; 7(9): e1002253. https://doi.org/10.1371/journal pgen.1002253.
9. Ghazi A, Henis-Korenblit S, Kenyon C. Regulation of Caenorhabditis Elegans lifespan by a proteasomal E3 ligase complex. Proc Natl Acad Sci. 2007; 104(14):-47- 52. https://doi.org/10.1073/pnas.0700638104.
10. Jana N R. Protein homeostasis and aging: role of ubiquitin protein ligases. Neurochem Int. 2012; 60(5): 443-7. https://doi.org/10.1016/j.neuint.2012.02.009.
11. López-Otín C, Blasco MA, Partridge L, Serrano M, Kroemer G. The hallmarks of aging. Cell. 2013; 153(6):-94- 217. https://doi.org/10.1016/j.cell.2013.05.039.
12. López-Otín , C, Blasco, M, Partridge, L, Serrano, M, Kroemer, G. 2023 Hallmarks of aging: an expanding universe. Cell 186(2) https://doi.org/10.1016/j.cell.2022.11.001.
13. Jensen M B, Jasper H. Mitochondrial proteostasis in the control of aging and longevity. Cell Metab. 2014; 20(2): 214-25. https://doi.org/10.1016/j.cmet.2014.05.006.
14. Rodriguez-FernandezIA, QiY, JasperH. Lossofaproteostatic checkpoint in intestinal stem cells contributes to age-related epithelial dysfunction. Nat Commun. 2019; 10(1): 1050. https://doi.org/10.1038/s41467-019-08982-9.
15. Wang L, Davis S S, Borch Jensen M, Rodriguez-Fernandez I A, Apaydin C, Juhasz G, Gibson B W, Schilling B, Ramanathan A, Ghaemmaghami S, Jasper H. JNK modifies neuronal metabolism to promote proteostasis and longevity. Aging Cell. 2019; 18(3): e12849. https://doi.org/10.1111/acel.12849.
16. Ameri K, Harris AL. Activating transcription factor 4. Int J Biochem Cell Biol. 2008; 40(1): 14-21. https://doi.org/10.1016/j.biocel.2007.01.020.
17. Hinnebusch AG, Natarajan K. Gcn4 p, a master regulator of gene expression, is controlled at multiple levels by diverse signals of starvation and stress. Eukaryot Cell. 2002; 1(1):22-32. https://doi.org/10.1128/EC.01.1.22-32.2002.
18. McCormick M A, Delaney J R, Tsuchiya M, Tsuchiyama S, Shemorry A, Sim S, Chou A C-Z, Ahmed U, Carr D, Murakami C J, Schleit J, Sutphin G L, Wasko B M, Bennett C F, Wang A M, Olsen B, Beyer R P, Bammler T K, Prunkard D, Johnson S C, Pennypacker J K, An E, Anies A, Castanza A S, Choi E, Dang N, Enerio S, Fletcher M, Fox L, Goswami S, Higgins SA, Holmberg M A, Hu D, Hui J, Jelic M, Jeong K-S, Johnston E, Kerr E O, Kim J, Kim D, Kirkland K, Klum S, Kotireddy S, Liao E, Lim M, Lin M S, Lo W C, Lockshon D, Miller H A, Moller RM , Muller B, Oakes J, Pak D N, Peng Z J, Pham K M, Pollard T G, Pradeep P, Pruett D, Rai D, Robison B, Rodriguez A A, Ros B, Sage M, Singh M K, Smith E D, Snead K, Solanky A, Spector B L, Steffen K K, Tchao B N, Ting M K, Vander Wende H, Wang D, Welton K L, Westman E A, Brem R B, Liu X, Suh Y, Zhou Z, Kaeberlein M, Kennedy B K. A Comprehensive analysis of replicative lifespan in 4,698 single-gene deletion strains uncovers conserved mechanisms of aging. Cell Metab. 2015; 22(5): 895-906. https://doi.org/10.1016/j.cmet.2015.09.008.
19. Ramirez M, Wek R C, Vazquez de Aldana C R, Jackson B M, Freeman B, Hinnebusch A G. Mutations activating the yeast EIF-2 alpha kinase GCN2: Isolation of alleles altering the domain related to Histidyl-TRNA synthetases. Mol Cell Biol. 1992; 12(12): 5801-15. https://doi.org/10.1128/mcb.12.12.5801-5815.1992.
20. Hinnebusch A G. Translational regulation of GCN4 and the general amino acid control of yeast. Annu Rev Microbiol. 2005; 59:407-50. https://doi.org/10.1146/ annurev.micro.59.031805.133833.
21. Molenaars M, Janssens G E, Williams E G, Jongejan A, Lan J, Rabot S, Joly F, Moerland PD, Schomakers B V, Lezzerini M, Liu Y J, McCormick M A, Kennedy B K, van Weeghel M, van Kampen A H C, Aebersold R, MacInnes A W, Houtkooper R H. A Conserved mito-cytosolic translational balance links two longevity pathways. Cell Metab. 2020; 31(3): 549-563. e7. https://doi.org/10.1016/j.cmet. 2020.01.011.
22. Wei N, Zhu L-Q, Liu D. ATF4: a novel potential therapeutic target for Alzheimer's disease. Mol Neurobiol. 2015; 52(3):-65- 70. https://doi.org/10.1007/s 12035-014-8970-8.
23. Kitakaze K, Oyadomari M, Zhang J, Hamada Y, Takenouchi Y, Tsuboi K, Inagaki M, Tachikawa M, Fujitani Y, Okamoto Y, Oyadomari S. ATF4-mediated transcriptional regulation protects against β-cell loss during endoplasmic reticulum stress in a mouse model. Mol Metab. 2021; 54:101338. https://doi.org/10.1016/j.molmet.2021.101338.
24. Harding H P, Zhang Y, Zeng H, Novoa I, Lu P D, Calfon M, Sadri N, Yun C, Popko B, Paules R, Stojdl DF, Bell J C, Hettmann T, Leiden J M, Ron D. an integrated stress response regulates amino acid metabolism and resistance to oxidative stress. Mol Cell. 2003; 11(3): 619-33. https://doi.org/10.1016/S 1097-2765(03)00105-9.
25. Tian X, Zhang S, Zhou L, Seyhan A A, Hernandez Borrero L, Zhang Y, El-Deiry W S. Targeting the integrated stress response in cancer therapy. Front Pharmacol. 2021; 12:747837. https://doi.org/10.3389/fphar.2021.747837.
26. Ebert S M, Dyle M C, Bullard S A, Dierdorff J M, Murry D J, Fox D K, Bongers K S, Lira V A, Meyerholz D K, Talley J J, Adams C M. Identification and small molecule inhibition of an activating transcription factor 4 (ATF4) dependent pathway to age-related skeletal muscle weakness and atrophy. J Biol Chem. 2015; 290(42):-497- 511. https://doi.org/10.1074/jbc. M115.681445.
27. Robbins C E, Patel B, Sawyer DL, Wilkinson B, Kennedy B K, McCormick M A. Cytosolic and mitochondrial TRNA synthetase inhibitors increase lifespan in a GCN4/Atf-4-dependent manner. iScience. 2022; 25(11): 105410. https://doi.org/10.1016/j.isci.2022.105410.
28. Hu Z, Xia B, Postnikoff S D, Shen Z-J, Tomoiaga A S, Harkness T A, Seol J H, Li W, Chen K, Tyler JK. Ssd1 and Gcn2 suppress global translation efficiency in replicatively aged yeast while their activation extends lifespan. eLife. 2018; 7: e35551. https://doi.org/10.7554/eLife.35551.
29. Statzer C, Meng J, Venz R, Bland M, Robida-Stubbs S, Patel K, Petrovic D, Emsley R, Liu P, Morantte I, Haynes C, Mair W B, Longchamp A, Filipovic M R, Blackwell T K, Ewald CY. ATF-4 and hydrogen sulfide signalling mediate longevity in response to inhibition of translation or MTORC1. Nat Commun. 2022; 13(1): 967. https://doi.org/10.1038/s41467-022-28599-9.
30. Steffen K K, MacKay V L, Kerr E O, Tsuchiya M, Hu D, Fox LA, Dang N. Johnston E D, Oakes J A, Tchao B N, Pak D N, Fields S, Kennedy B K, Kaeberlein M. Yeast life span extension by depletion of 60S ribosomal subunits is mediated by Gcn4. Cell. 2008; 133(2): 292-302. https://doi.org/10.1016/j.cell.2008.02.037.
31. Kang M-J, Vasudevan D, Kang K, Kim K, Park J-E, Zhang N, Zeng X, Neubert T A, Marr MT, Ryoo HD. 4E-BP is a target of the GCN2-ATF4 pathway during Drosophila development and aging. J Cell Biol. 2017; 216(1): 115-29. https://doi.org/10.1083/jcb.201511073.
32. Li W, Li X, Miller R A. ATF4 Activity: a common feature shared by many kinds of slow-aging mice. Aging Cell. 2014; 13(6): 1012-8. https://doi.org/10.1111/acel.12264.
33. Li W, Miller R A. Elevated ATF4 function in fibroblasts and liver of slow-aging mutant mice. J Gerontol Ser A. 2015; 70(3): 263-72. https://doi.org/10.1093/gerona/glu 040.
34. Hinnebusch AG. The scanning mechanism of eukaryotic translation initiation. Annu Rev Biochem. 2014; 83(1): 779-812. https://doi.org/10.1146/annurev-biochem-060713-035802.
35. Kilberg M S, Shan J, Su N. ATF 4-dependent transcription mediates signaling of amino acid limitation. Trends Endocrinol Metab. 2009; 20(9): 436-43. https://doi.org/ 10.1016/j.tem.2009.05.008.
36. Lu P D, Harding H P, Ron D. Translation reinitiation at alternative open reading frames regulates gene expression in an integrated stress response. J Cell Biol. 2004; 167(1): 27-33. https://doi.org/10.1083/jcb.200408003.
37. Harding H P, Novoa I, Zhang Y, Zeng H, Wek R, Schapira M, Ron D. Regulated translation initiation controls stress-induced gene expression in mammalian cells. Mol Cell. 2000; 6(5): 1099-108. https://doi.org/10.1016/S 1097-2765(00)00108-8.
38. Nakatogawa H, Suzuki K, Kamada Y, Ohsumi Y. Dynamics and diversity in autophagy mechanisms: lessons from yeast. Nat Rev Mol Cell Biol. 2009; 10(7):-8-67. https://doi.org/10.1038/nrm2708.
39. Wong S Q, Kumar A V, Mills J, Lapierre L R. Autophagy in aging and longevity. Hum Genet. 2020; 139(3): 277-90. https://doi.org/10.1007/s 00439-019-02031-7.
40. Rubinsztein D C, Mariño G, Kroemer G. Autophagy and aging. Cell. 2011; 146(5):-2- 95. https://doi.org/10. 1016/j.cell.2011.07.030.
41. Cebollero E, Reggiori F, Kraft C. Reticulophagy and ribophagy: regulated degradation of protein production factories. Int J Cell Biol. 2012; 2012: e182834. https://doi.org/10.1155/2012/182834.
42. Tolkovsky A M. Mitophagy. Biochim Biophys Acta BBA Mol Cell Res. 2009;1793(9): 1508-15. https://doi.org/10.1016/j.bbamcr.2009.03.002.
43. Zaffagnini G, Martens S. Mechanisms of selective autophagy. J Mol Biol. 2016; 428(9 Part A): 1714-24. https://doi.org/10.1016/j.jmb.2016.02.004.
44. Tóth M L, Sigmond T, Borsos É, Barna J, Erdélyi P, Takács-Vellai K, Orosz L, Kovács A L, Csikós G, Sass M, Vellai T. Longevity pathways converge on autophagy genes to regulate life span in Caenorhabditis Elegans. Autophagy. 2008; 4(3): 330-8. https://doi.org/10.4161/auto.5618.
45. Bjedov I, Toivonen J M, Kerr F, Slack C, Jacobson J, Foley A, Partridge L. Mechanisms of life span extension by rapamycin in the fruit fly Drosophila Melanogaster. Cell Metab. 2010; 11(1): 35-46. https://doi.org/10.1016/j.cmet.2009.11.010.
46. Hansen M, Chandra A, Mitic L L, Onken B, Driscoll M, Kenyon C. A role for autophagy in the extension of lifespan by dietary restriction in C. Elegans PLOS Genet. 2008; 4(2): e24. https://doi.org/10.1371/journal.pgen. 0040024. GeroScience
47. ShenZ-J, PostnikoffS, Tyler J K. IsGcn4-inducedautophagythe ultimate downstream mechanism by which hormesis extends yeast replicative lifespan? Curr Genet. 2019; 65(3): 717-20. https://doi.org/10.1007/s 00294-019-00936-4.
48. Alvers AL, Fishwick L K, Wood M S, Hu D, Chung HS, Dunn W A, Aris JP. Autophagy and amino acid homeostasis are required for chronological longevity in Saccharomyces Cerevisiae. Aging Cell. 2009; 8(4): 353-69. https://doi.org/10.1111/j.1474-9726.2009.00469. x.
49. Yamamoto H, Zhang S, Mizushima N. Autophagy genes in biology and disease. Nat Rev Genet. 2023; 24(6):-2- 400. https://doi.org/10.1038/s 41576-022-00562-w.
50. B'chir W, Maurin A-C, Carraro V, Averous J, Jousse C, Muranishi Y, Parry L, Stepien G, Fafournoux P, Bruhat A. The EIF2α/ATF4 pathway is essential for stress-induced autophagy gene expression. Nucleic Acids Res. 2013; 41(16): 7683-99. https://doi.org/10.1093/nar/gkt563.
51. Vallejo M, Ron D, Miller C P, Habener J F. C/ATF, a member of the activating transcription factor family of dna-binding proteins, dimerizes with CAAAT/enhancer-binding proteins and directs their binding to camp response elements. Proc Natl Acad Sci U S A. 1993; 90(10): 4679-83. https://doi.org/10.1073/pnas.90. 10.4679.
52. Rzymski T, Milani M, Pike L, Buffa F, Mellor H R, Winchester L, Pires I, Hammond E, Ragoussis I, Harris AL. Regulation of autophagy by ATF4 in response to severe hypoxia. Oncogene. 2010; 29(31): 4424-35. https://doi.org/10.1038/onc.2010.191.
53. Verginadis I I, Avgousti H, Monslow J, Skoufos G, Chinga F, Kim K, Leli N M, Karagounis I V, Bell B I, Velalopoulou A, Salinas C S, Wu V S, Li Y, Ye J, Scott D A, Osterman A L, Sengupta A, Weljie A, Huang M, Zhang D, Fan Y, Radaelli E, Tobias J W, Rambow F, Karras P, Marine J-C, Xu X, Hatzigeorgiou AG, Ryeom S, Diehl JA, Fuchs S Y, Puré E, Koumenis C. A stromal integrated stress response activates perivascular cancer-associated fibroblasts to drive angiogenesis and tumour progression. Nat Cell Biol. 2022; 24(6): 940-53. https://doi.org/10.1038/s 41556-022-00918-8.
54. D'Aniello C, Fico A, Casalino L, Guardiola O, Di Napoli G, Cermola F, De Cesare D, Taté R, Cobellis G, Patriarca E J, Minchiotti G. A novel autoregulatory loop between the Gcn2-Atf4 pathway and L-proline metabolism controls stem cell identity. Cell Death Differ. 2015; 22(7): 1094-105. https://doi.org/10.1038/cdd. 2015.24.
55. Rolfes R J, Hinnebusch A G. Translation of the yeast transcriptional activator GCN4 is stimulated by purine limitation: implications for activation of the protein kinase GCN2. Mol Cell Biol. 1993; 13(8): 5099-111. https://doi.org/10.1128/mcb.13.8.5099-5111.1993.
56. Kroemer G, Mariño G, Levine B. Autophagy and the integrated stress response. Mol Cell. 2010; 40(2): 280-93. https://doi.org/10.1016/j.molcel.2010.09.023.
57. Deegan S, Koryga I, Glynn SA, Gupta S, Gorman A M, Samali A close connection between the PERK and IRE arms of the UPR and the transcriptional regulation of autophagy. Biochem Biophys Res Commun. 2015; 456(1): 305-11. https://doi.org/10.1016/j.bbrc.2014.11.076.
58. Milano A, Iaffaioli R V, Caponigro F. 2007 The proteasome: a worthwhile target for the treatment of solid tumours? Eur J Cancer Oxf Engl. 1990; 43(7): 1125-33. https://doi.org/10.1016/j.ejca.2007.01.038.
59. KisselevA F, GoldbergA L. Proteasomeinhibitors: fromresearch tools to drug candidates. Chem Biol. 2001; 8(8): 739-58. https://doi.org/10.1016/s 1074-5521(01)00056-4.
60. Frake R A, Ricketts T, Menzies F M, Rubinsztein D C. Autophagy and neurodegeneration. J Clin Invest. 2015; 125(1): 65-74. https://doi.org/10.1172/JCI 73944.
61. Jia K, Levine B. Autophagy is required for dietary restriction-mediated life span extension in C. Elegans Autophagy. 2007; 3(6): 597-9. https://doi.org/10.4161/auto.4989.
62. David, D. Aging and the Aggregating Proteome. Front Genet 2012; 3.
63. David D C, Ollikainen N, Trinidad J C, Cary M P, Burlingame A L, Kenyon C. Widespread protein aggregation as an inherent part of aging in C Elegans. PLOS Biol. 2010; 8(8): e1000450. https://doi.org/10.1371/journal.pbio.1000450.
64. Xu, J. Preparation, culture, and immortalization of mouse embryonic fibroblasts. Curr Protoc Mol Biol Chapter 2005; 28(Unit 28.1) https://doi.org/10.1002/04711 42727. mb2801s70.
65. Qiu, L., Lai, W., Stumpo, D., Blackshear, P. Mouse embryonic fibroblast cell culture and stimulation. BIOProtoc. 2016; 6(13) https://doi.org/10.21769/BioProtoc. 1859.
66. Sieber J, Hauer C, Bhuvanagiri M, Leicht S, Krijgsveld J, Neu-Yilik G, Hentze M W, Kulozik A E. Proteomic Analysis reveals branch-specific regulation of the unfolded protein response by nonsense-mediated MRNA decay. Mol Cell Proteomics. 2016; 15(5): 1584-97. https://doi.org/10.1074/mcp. M 115.054056.
67. Miyake M, Nomura A, Ogura A, Takehana K, Kitahara Y, Takahara K, Tsugawa K, Miyamoto C, Miura N, Sato R, Kurahashi K, Harding HP, Oyadomari M, Ron D, Oyadomari S. Skeletal muscle-specific eukaryotic translation initiation factor 2α Phosphorylation controls amino acid metabolism and fibroblast growth factor 21-mediated non-cell-autonomous energy metabolism. FASEB J Off Publ Fed Am Soc Exp Biol. 2016; 30(2): 798-812. https://doi.org/10.1096/fj.15-275990.
68. Schipper-Krom, S.; Sanz, A. S.; van Bodegraven, E. J.; Speijer, D.; Florea, B. I.; Ovaa, H.; Reits, E. A. Visualizing Proteasome Activity and Intracellular Localization Using Fluorescent Proteins and Activity-Based Probes. Front Mol Biosci 2019; 6.
69. Kisselev A F, Garcia-Calvo M, Overkleeft H S, Peterson E, Pennington M W, Thornberry N A, Goldberg A L, Ploegh H L. the caspase-like sites of proteasomes, their substrate specificity, new inhibitors and substrates, and allosteric interactions with the trypsin-like sites. J Biol Chem. 2003; 278(38): 35869-77. https://doi.org/10.1074/jbc. M303725200.
70. Kaizuka T, Morishita H, Hama Y, Tsukamoto S, Matsui T, Toyota Y, Kodama A, Ishihara T, Mizushima T, Mizushima N. An autophagic flux probe that releases an internal control. Mol Cell. 2016; 64(4): 835-49. https://doi.org/10.1016/j.molcel.2016.09.037.
71. Kimura S, Noda T, Yoshimori T. Dissection of the autophagosome maturation process by a novel reporter protein, tandem fluorescent-tagged LC3. Autophagy. 2007; 3(5): 452-60. https://doi.org/10.4161/auto.4451.
72. Chung M, Bruno V M, Rasko DA, Cuomo CA, Muñoz J F, Livny J, Shetty A C, Mahurkar A, Dunning Hotopp JC. Best practices on the differential expression analysis of multi-species RNA-seq. Genome Biol. 2021; 22(1): 121. https://doi.org/10.1186/s13059-021-02337-8.
73. Liao Y, Smyth G K, Shi W. FeatureCounts: an efficient general purpose program for assigning sequence reads to genomic features. Bioinformatics. 2014; 30(7): 923-30. https://doi.org/10.1093/bioinformatics/btt656.
74. Chen S, Zhou Y, Chen Y, Gu J. Fastp: An ultra-fast all-in-one FASTQ preprocessor. Bioinformatics. 2018; 34(17):-84- 90. https://doi.org/10.1093/bioinformatics/bty560.
75. Kim D, Paggi J M, Park C, Bennett C, Salzberg S L. Graphbased genome alignment and genotyping with HISAT 2 and HISAT-genotype. Nat Biotechnol. 2019; 37(8): 907-15. https://doi.org/10.1038/s41587-019-0201-4.
76. Law C W, Chen Y, Shi W, Smyth G K. Voom: precision weights unlock linear model analysis tools for RNA-seq read counts. Genome Biol. 2014; 15(2): R29. https://doi.org/10.1186/gb-2014-15-2-r29.
77. Smyth, G. K. Limma: linear models for microarray data. In Bioinformatics and Computational Biology Solutions Using R and Bioconductor; Gentleman, R., Carey, V. J., Huber, W., Irizarry, R. A., Dudoit, S., Eds.; Statistics for Biology and Health; Springer: New York, NY, 2005; 397-420. https://doi.org/10.1007/0-387-29362-0_23.
78. Aldridge G M, Podrebarac D M, Greenough W T, Weiler I J. The use of total protein stains as loading controls: an alternative to high-abundance single-protein controls in semi-quantitative immunoblotting. J Neurosci Methods. 2008; 172(2): 250-4. https://doi.org/10.1016/j.jneumeth.2008.05.003.
79. Berger J, Jampolsky L M, Goldberg M W. Borrelidin, a new antibiotic with antiborrelia activity and penicillin enhancement properties. Arch Biochem. 1949; 22(3): 476-8.
80. Habibi D, Ogloff N, Jalili R B, Yost A, Weng AP, Ghahary A, Ong CJ. Borrelidin, a small molecule nitrile-containing macrolide inhibitor of threonyl-TRNA synthetase, is a potent inducer of apoptosis in acute lymphoblastic leukemia. Invest New Drugs. 2012; 30(4): 1361-70. https://doi.org/10.1007/s 10637-011-9700-y.
81. Kitajima S, Sun W, Lee K L, Ho JC , Oyadomari S, Okamoto T, Masai H, Poellinger L, Kato H. A KDM6 inhibitor potently induces ATF4 and its target gene expression through HRI activation and by UTX inhibition. Sci Rep. 2021; 11(1): 4538. https://doi.org/10.1038/s41598-021-83857-y.
82. Bouz G, Zitko J. Inhibitors of aminoacyl-TRNA synthetases as antimycobacterial compounds: an up-to-date review. Bioorganic Chem. 2021; 110:104806. https://doi.org/10.1016/j.bioorg.2021.104806.
83. Capobianco J O, Doran C C, Goldman R C. Mechanism of mupirocin transport into sensitive and resistant bacteria. Antimicrob Agents Chemother. 1989; 33(2): 156-63.
84. Baragaña B, Forte B, Choi R, Nakazawa Hewitt S, Bueren-Calabuig J A, Pisco J P, Peet C, Dranow D M, Robinson D A, Jansen C, Norcross N R, Vinayak S, Anderson M, Brooks C F, Cooper C A, Damerow S, Delves M, Dowers K, Duffy J, Edwards T E, Hallyburton I, Horst B G, Hulverson M A, Ferguson L, Jiménez-Díaz M B, Jumani R S, Lorimer D D, Love M S, Maher S, Matthews H, McNamara C W, Miller P, O'Neill S, Ojo K K, Osuna-Cabello M, Pinto E, Post J, Riley J, Rottmann M, Sanz L M, Scullion P, Sharma A, Shepherd S M, Shishikura Y, Simeons F R C, Stebbins E E, Stojanovski L, Straschil U, Tamaki F K, Tamjar J, Torrie L S, Vantaux A, Witkowski B, Wittlin S, Yogavel M, Zuccotto F, Angulo-Barturen I, Sinden R, Baum J, Gamo F-J, Mäser P, Kyle D E, Winzeler E A, Myler P J, Wyatt P G, Floyd D, Matthews D, Sharma A, Striepen B, Huston CD, Gray D W, Fairlamb A H, Pisliakov A V, Walpole C, Read K D, Van Voorhis W C, Gilbert I H. Lysyl-TRNA synthetase as a drug target in malaria and Cryptosporidiosis. Proc Natl Acad Sci U S A. 2019; 116(14): 7015-20. https://doi.org/10.1073/pnas.1814685116.
85. Critchley I A, Young C L, Stone K C, Ochsner U A, Guiles J, Tarasow T, Janjic N. Antibacterial Activity of REP8839, a new antibiotic for topical use. Antimicrob Agents Chemother. 2005; 49(10): 4247-52. https://doi.org/10.1128/AAC.49.10.4247-4252.2005.
86. Critchley I A, Green L S, Young C L, Bullard J M, Evans R J, Price M, Jarvis T C, Guiles J W, Janjic N, Ochsner U A. Spectrum of activity and mode of action of REP3123, a new antibiotic to treat Clostridium Difficile Infections. J Antimicrob Chemother. 2009; 63(5): 954-63. https://doi.org/10.1093/jac/dkp 041.
87. Mazor K M, Stipanuk M H. GCN 2-and EIF 2α-phosphorylation-independent, but ATF 4-dependent, induction of CARE-containing genes in methionine-deficient cells. Amino Acids. 2016; 48(12): 2831-42. https://doi.org/10.1007/s 00726-016-2318-9.
88. McCormick M, Chen K, Ramaswamy P, Kenyon C. New genes that extend Caenorhabditis Elegans'lifespan in response to reproductive signals. Aging Cell. 2012; 11(2): 192-202. https://doi.org/10.1111/j.1474-9726.2011.00768. x.
89. Heinz S, Benner C, Spann N, Bertolino E, Lin YC, Laslo P, Cheng J X, Murre C, Singh H, Glass C K. Simple combinations of lineage-determining transcription factors prime Cis-regulatory elements required for macrophage and B cell identities. Mol Cell. 2010; 38(4): 576-89. https://doi.org/10.1016/j.molcel.2010.05.004.
90. Bindea G, Mlecnik B, Hackl H, Charoentong P, Tosolini M, Kirilovsky A, Fridman W-H, Pagès F, Trajanoski Z, Galon J. ClueGO: A cytoscape plug-in to decipher functionally grouped gene ontology and pathway annotation networks. Bioinforma Oxf Engl. 2009; 25(8): 1091-3. https://doi.org/10.1093/bioinformatics/btp 101.
91. Mittal N, Guimaraes J C, Gross T, Schmidt A, Vina-Vilaseca A, Nedialkova D D, Aeschimann F, Leidel SA, Spang A, Zavolan M. The Gcn4 transcription factor reduces protein synthesis capacity and extends yeast lifespan. Nat Commun. 2017; 8(1): 457. https://doi.org/10. 1038/s41467-017-00539-y.
92. Dooley H C, Razi M, Polson H E J, Girardin S E, Wilson M I, Tooze S A. WIPI2 links LC3conjugation with PI3P, autophagosome formation, and pathogen clearance by recruiting Atg 12-5-16L1. Mol Cell. 2014; 55(2): 238-52. https://doi.org/10.1016/j.molcel.2014.05.021.
93. Wilson M I, Dooley HC, Tooze SA. WIPI2 b and Atg 16L1: setting the stage for autophagosome formation. GeroScience Biochem Soc Trans. 2014; 42(5): 1327-34. https://doi.org/10.1042/BST20140177.
94. Torrence M E, MacArthur M R, Hosios A M, Valvezan A J, Asara J M, Mitchell J R, Manning B D. The MTORC1-mediated activation of ATF4 promotes protein and glutathione synthesis downstream of growth signals. eLife. 2021; 10: e63326. https://doi.org/10.7554/eLife.63326.
95. Harding H P, Zhang Y, Bertolotti A, Zeng H, Ron D. Perk is essential for translational regulation and cell survival during the unfolded protein response. Mol Cell. 2000; 5(5):-7- 904. https://doi.org/10.1016/S 1097-2765(00)80330-5.
96. Tameire F, Verginadis II, Leli N M, Polte C, Conn C S, Ojha R, Salas Salinas C, Chinga F, Monroy A M, Fu W, Wang P, Kossenkov A, Ye J, Amaravadi RK, Ignatova Z, Fuchs SY, Diehl JA, Ruggero D, Koumenis C. ATF4 Couples MYC-dependent translational activity to bioenergetic demands during tumour progression. Nat Cell Biol. 2019; 21(7): 889-99. https://doi.org/10.1038/s 41556-019-0347-9.
97. Jiang D, Cui H, Xie N, Banerjee S, Liu R-M, Dai H, Thannickal VJ, Liu G. ATF4Mediates mitochondrial unfolded protein response in alveolar epithelial cells. Am J Respir Cell Mol Biol. 2020; 63(4): 478-89. https://doi.org/10.1165/rcmb.2020-0107OC.
98. Hu J, Dang N, Menu E, De Bruyne E, Xu D, Van Camp B, Van Valckenborgh E, Vanderkerken K. Activation of ATF4 mediates unwanted Mcl-1 accumulation by proteasome inhibition. Blood. 2012; 119(3): 826-37. https://doi.org/10.1182/blood-2011-07-366492.
99. Lassot I, Ségéral E, Berlioz-Torrent C, Durand H, Groussin L, Hai T, Benarous R, Margottin-Goguet F. ATF4 degradation relies on a phosphorylation-dependent interaction with the SCF(BetaTrCP) ubiquitin ligase. Mol Cell Biol. 2001; 21(6):-92-202. https://doi.org/10.1128/MCB.21.6.2192-2202.2001.
100. Zou K, Rouskin S, Dervishi K, McCormick M A, Sasikumar A, Deng C, Chen Z, Kaeberlein M, Brem R B, Polymenis M, Kennedy B K, Weissman JS, Zheng J, Ouyang Q, Li H. Life span extension by glucose restriction is abrogated by methionine supplementation: cross-talk between glucose and methionine and implication of methionine as a key regulator of life span. Sci Adv. 2020; 6(32): eaba1306. https://doi.org/10.1126/sciadv. aba1306.
101. Adams J. The development of proteasome inhibitors as anticancer drugs. Cancer Cell. 2004; 5(5): 417-21. https://doi.org/10.1016/s 1535-6108(04)00120-5.
102. Kane R C, Bross P F, Farrell A T, Pazdur R. Velcade: U.S. FDA approval for the treatment of multiple myeloma progressing on prior therapy. Oncologist. 2003; 8(6):-8- 13. https://doi.org/10.1634/theoncologist.8-6-508.
103. Nieto-Torres J L, Hansen M. Macroautophagy and aging: the impact of cellular recycling on health and longevity. Mol Aspects Med. 2021; 82:101020. https://doi.org/10. 1016/j.mam.2021.101020.
104. Levine B, Packer M, Codogno P. Development of autophagy inducers in clinical medicine. J Clin Invest. 2015; 125(1): 14-24. https://doi.org/10.1172/JCI 73938.
105. Zhang Z D, Milman S, Lin J-R, Wierbowski S, Yu H, Barzilai N, Gorbunova V, Ladiges WC, Niedernhofer LJ, Suh Y, Robbins PD, Vijg J. Genetics of extreme human longevity to guide drug discovery for healthy ageing. Nat Metab. 2020; 2(8):-3-72. https://doi.org/10.1038/s42255-020-0247-0.
106. Ron D, Walter P. signal integration in the endoplasmic reticulum unfolded protein response. Nat Rev Mol Cell Biol. 2007; 8(7): 519-29. https://doi.org/10.1038/nrm 2199.
107. Patil C K, Li H, Walter P. Gcn4 p and novel upstream activating sequences regulate targets of the unfolded protein response. PLoS Biol. 2004; 2(8): e246. https://doi.org/10. 1371/journal pbio.0020246.
108. Woo CW, Cui D, Arellano J, Dorweiler B, Harding H, Fitzgerald K A, Ron D, Tabas I. Adaptive suppression of the ATF4-CHOP branch of the unfolded protein response by Toll-like receptor signalling. Nat Cell Biol. 2009; 11(12): 1473-80. https://doi.org/10.1038/ncb1996.
109. Fusakio ME, Willy J A, Wang Y, Mirek E T, Al Baghdadi R J T, Adams C M, Anthony T G, Wek R C. Transcription factor ATF4 directs basal and stress-induced gene expression in the unfolded protein response and cholesterol metabolism in the liver. Mol Biol Cell. 2016; 27(9):-36-51. https://doi.org/10.1091/mbc. E 16-01-0039.
110. Solis G M, Kardakaris R, Valentine E R, Bar-Peled L, Chen A L, Blewett M M, McCormick M A, Williamson J R, Kennedy B, Cravatt B F, Petrascheck M. Translation attenuation by minocycline enhances longevity and proteostasis in old post-stress-responsive organisms.eLife. 2018; 7: e40314. https://doi.org/10.7554/eLife.40314.
111. Miller B F, Drake J C, Naylor B, Price J C, Hamilton K L. The measurement of protein synthesis for assessing proteostasis in studies of slowed aging. Ageing Res Rev. 2014;-:106- 11. https://doi.org/10.1016/j.arr.2014.09.005.
112. Zhu S, Sobolev A Y, Wek R C. Histidyl-TRNA synthetase-related sequences in GCN 2 protein kinase regulate in vitro phosphorylation of EIF-2. J Biol Chem. 1996; 271(40): 24989-94. https://doi.org/10.1074/jbc.271. 40.24989.
113. Dong J, Qiu H, Garcia-Barrio M, Anderson J, Hinnebusch A G. Uncharged TRNA activates GCN2 by displacing the protein kinase moiety from a bipartite TRNA-binding domain. Mol Cell. 2000; 6(2): 269-79. https://doi org/10.1016/s 1097-2765(00)00028-9.
114. Wek R C, Jackson B M, Hinnebusch A G. Juxtaposition of domains homologous to protein kinases and Histidyl-TRNA synthetases in GCN2 protein suggests a mechanism for coupling GCN4 expression to amino acid availability. Proc Natl Acad Sci. 1989; 86(12): 4579-83. https://doi.org/10.1073/pnas.86.12.4579.
115. Wek SA, Zhu S, Wek R C. The Histidyl-TRNA synthetase-related sequence in the EIF-2 alpha protein kinase GCN2 interacts with TRNA and is required for activation in response to starvation for different amino acids. Mol Cell Biol. 1995; 15(8):-97-506.
116. Pakos-Zebrucka K, Koryga I, Mnich K, Ljujic M, Samali A, Gorman A M. The integrated stress response. EMBO Rep. 2016; 17(10): 1374-95. https://doi.org/10.15252/embr.201642195.
117. Park Y, Reyna-Neyra A, Philippe L, Thoreen C C. MTORC1 balances cellular amino acid supply with demand for protein synthesis through post-transcriptional control of ATF 4. Cell Rep. 2017; 19(6): 1083-90. https://doi.org/10.1016/j.celrep.2017.04.042.

1. A method of treating, inhibiting, ameliorating, delaying the onset of or reducing the likelihood of a neurodegenerative disease in a patient in need or at risk comprising administering at least one tRNA synthetase inhibitor to the patient.

2. The method according to claim 1 wherein said neurodegenerative disease is a dementia-type diseases, demyelinating disease, a parkinsonism-type disease, a motor neuron disease, a tauopathy or a prion disease.

3. The method according to claim 1 or 2 wherein said neurodegenerative disease is Alzheimer's disease, frontotemporal dementia, chronic traumatic encephalopathy (CTE), Lewy body dementia, limbic predominant age-related TDP-43 encephalopathy (LATE), multiple sclerosis (MS), multiple system atrophy and neuromyletis optica spectrum disorder (NMOSD), Parkinson's disease, Huntington's disease, amyotrophic lateral sclerosis (ALS), progressive supranuclear palsy (PSP), Pick's disease, corticobasal degeneration, Creutzfeldt-Jacob disease (CJD), variant Creutzfeldt-Jacob disease (VCJD), variably protease-sensitive prionopathy (VPSPr), Gerstmann-Sträussler-Scheinker disease (GSS), Kuru or Fatal insomnia (FI).

4. The method according to claim 2 wherein said neurodegenerative disease is Alzheimer's disease, Parkinson's disease, Huntington's disease, amyotrophic lateral sclerosis (ALS) or multiple sclerosis.

5. The method according to any one of claims 1-4 wherein said tRNA synthetase inhibitor is borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, or a pharmaceutically acceptable salt thereof.

6. The method according to any one of claims 1-4 wherein said tRNA synthetase inhibitor is borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132), REP8839, or a pharmaceutically acceptable salt thereof.

7. The method according to any one of claims 1-6 wherein said tRNA synthetase inhibitor is co-administered with an additional bioactive agent.

8. The method according to claim 7 wherein said additional bioactive agent is selected from the group consisting of aducanumab, solanezumab, semorinemab, verubecestate, semagacesta (for AD), prasinezumab, affitope PD01a, a mGluR5 antagonist, a mGlur agonist, a mGlur4 agonist, SV2C and LRRK2 or a pharmaceutically acceptable salt thereof.

9. A pharmaceutical composition comprising an effective amount of a tRNA synthetase inhibitor and at least one additional bioactive agent.

10. The composition according to claim 9 wherein said tRNA synthetase inhibitor is borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132) REP8839, ganfeborole, PT638, PT662, GSK656, or a pharmaceutically acceptable salt thereof.

11. The composition according to claim 9 wherein said tRNA synthetase inhibitor is borrelidin, mupirocin, tavaborole (AN2690), halofuginone, LysRS-IN-2 (N-[(4,4-difluoro-1-hydroxycyclohexyl)methyl]-6-fluoro-4-oxochromene-2-carboxamide), cladosporin (asperentin), REP3132 (CRS-3132), REP8839, or a pharmaceutically acceptable salt thereof.

12. The composition according to any one of claims 9-11 wherein said additional bioactive agent is selected from the group consisting of aducanumab, solanezumab, semorinemab, verubecestate, semagacesta (for AD), prasinezumab, affitope PD01a, a mGluR5 antagonist, a mGlur agonist, a mGlur4 agonist, SV2C and LRRK2 or a pharmaceutically acceptable salt thereof.
